SEQUENCE LISTING (48 sequences)

SEQ ID NO: 1 - protein, 594 aa, synthetic construct
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVNAMAPSNFLATNPELLKLTLESDGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDAA

SEQ ID NO: 2 - protein, 594 aa, synthetic construct
MSQPSYGPLFEALAHYNDKLLAMAKAQTERTAQALLQTNLDDLGQVLEQGSQQPWQLIQAQMNWWQDQLKLMQHTLLKSAGQQSEPVITPERSDRRFKAEAWSEQPIYDYLKQSYLLTARHLLASVDALDGVPQKSRERLRFFTRQYVNAMAPSNFLATNPELLKLTLESDGQNLVRGLALLAEDLERSADQLNIRLTDESAFELGRDLATTPGRVVQRTELYELIQYSPTTETVGKTPVLIVPPFINKYYIMDMRPQNSLVAWLVAQGQTVFMISWRNPSVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPVANKYGFWHNGAEADSPESWLAGATHQSGSWWPEMMGFIQSRDEGSEPVPARVPEEGLAPAPGHYVKVRLNPVFASATEEDAA

SEQ ID NO: 3 - protein, 134 aa, synthetic construct
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 4 - protein, 134 aa, synthetic construct
MSAQPLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALRSDKPIATLTTRIFTSNGALAVTGEAVVKLP

SEQ ID NO: 5 - DNA, 55 bp, synthetic construct
  source 1..55: mol_type = other DNA
acctgtaggatcgtacaggtttacgcaagaaaatggtttgttactttcgaataaa

SEQ ID NO: 6 - DNA, 159 bp, synthetic construct
  source 1..159: mol_type = other DNA
ccgcttctagagctcggtaccaaattccagaaaagaggccgcgaaagcggccttttttcgttttggtcctactagatgcctccacaccgctcgtcacatcctgcccatgagttaattatatttgtggcattatagggagaattgtgagcgctcacaatt

SEQ ID NO: 7 - DNA, 43 bp, synthetic construct
  source 1..43: mol_type = other DNA
ttgcgttcactggaatcccannntagagtttgacctgcgagca

SEQ ID NO: 8 - DNA, 43 bp, synthetic construct
  source 1..43: mol_type = other DNA
ttgcgttcactggaatcccaatatagagtttgacctgcgagca

SEQ ID NO: 9 - DNA, 43 bp, synthetic construct
  source 1..43: mol_type = other DNA
ttgcgttcactggaatcccactatagagtttgacctgcgagca

SEQ ID NO: 10 - DNA, 43 bp, synthetic construct
  source 1..43: mol_type = other DNA
ttgcgttcactggaatcccagactagagtttgacctgcgagca

SEQ ID NO: 11 - DNA, 43 bp, synthetic construct
  source 1..43: mol_type = other DNA
ttgcgttcactggaatcccagtattaaatttgacctgcgagca

SEQ ID NO: 12 - DNA, 43 bp, synthetic construct
  source 1..43: mol_type = other DNA
ttgcgttcactggaatcccagtatctaatttgacctgcgagca

SEQ ID NO: 13 - DNA, 43 bp, synthetic construct
  source 1..43: mol_type = other DNA
ttgcgttcactggaatcccagtatgctatttgacctgcgagca

SEQ ID NO: 14 - DNA, 43 bp, synthetic construct
  source 1..43: mol_type = other DNA
ttgcgttcactggaatcccagtatcccttttgacctgcgagca

SEQ ID NO: 15 - DNA, 229 bp, synthetic construct
  source 1..229: mol_type = other DNA
taaccatctaatgatcagcgagtcatgctgggatggttaaacaagctgttaattagtctatttctccaatagtggcgcggctaaacttggactaaggccgcgcagtttgacgctaaaactactaaccgagacgctactcatgcagccaactgcacgcttttcattgtcgccggctcttcctgagacccacactcaacacagccatggagcttgctgttgtggttcacca

SEQ ID NO: 16 - DNA, 407 bp, synthetic construct
  source 1..407: mol_type = other DNA
gatggcaacgccccctctgtaatacctacgcatgaattactcctttagagcgtgtaaatcgccagcacaacgcccttatgaaaagacgcttaaatcgagcgtctgattaaaggccgatagcccaaagcaggctcgagcaatcagccttccacccaagctattttcgttaattctttaccccagaaaaactccacaaatacaaaatatagttaacttttgaggcgatgcaaatccaccttccgagcacatcgatcattcacctagctagatgagacaggcagctaatcatgtgcatcatcattaaaaattaaccgagtagcttgcggcacttcatctcaccttcatttccttatgctgaacacggtaagcttggcgataacaccgctactgatggctaaaaatcaacg

SEQ ID NO: 17 - DNA, 279 bp, synthetic construct
  source 1..279: mol_type = other DNA
aggtagtcgcattatcttcttttgtgctttcgccttatggctctaggcgcttggatgctcgtgatattttcctactcattggtcggaagattcgagactatcttcgatgtacaccattacgggggtgtcacggggtgatatagagtgtatcgcgcaaagttcacaaatgtgtcacggggactctaacgatagggcgatgtctgcattaacgcctcccctcatttcaataagtgtgtttcagcaaggagagcgcccatggcgactacacgccgtaaactc

SEQ ID NO: 18 - DNA, 376 bp, synthetic construct
  source 1..376: mol_type = other DNA
gtacgcagtaatgcgcgcactcagtgcacgactcttgtttaattaatcaaaagtaagaaactagttcttaaaaactaaccacattgttaaattctgtcatagcgactcagtgtaaaaactcattcaactaatcgcctacaacaaagacaagcttttatataccagcctttaaaaccgggcaactaaatatcacccaaggctttaaattaaacaataagaaacaattatcgcattaataattaacgctaaaaatcatgaattaattaaactgctaaaaagtgtttctttaacactttgatttttaattgactaataaaaaccattaaggaagcactaatgctacttaaaacgcccgctagcgattagcggtcatcgc

SEQ ID NO: 19 - DNA, 26 bp, synthetic construct
  source 1..26: mol_type = other DNA
tactagagaaagaggagaaatactag

SEQ ID NO: 20 - DNA, 26 bp, synthetic construct
  source 1..26: mol_type = other DNA
tactagagaaagaggagaaatactag

SEQ ID NO: 21 - DNA, 129 bp, synthetic construct
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 22 - DNA, 129 bp, synthetic construct
  source 1..129: mol_type = other DNA
ctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 23 - protein, 616 aa, synthetic construct
MLSGWKMPSQGVSQEELEAWKVQLSDVGEQYKGLLEDLLSRMVPSEAADSVQSDMRESFEAAAQSLMSNPNLLWQTQSRLLQDQWLLWQQGVRAMSGEQVTPLVTPAKGDRRFKDEAWTQEPYYLAIMQQYLLFSQMVEELIESLDDLDPTQKRNLAFYARQLVSAMSPTNFVSTNPEVMRCTLETRGQNLVDGLTRLREDLANSAEGINVRMTDRSAFGVGDNIAVTPGAVVYENELIQLIQYTPTTEKTFKTPLLIVPPWINKYYILDLREDNSLVKWMVDQGHTVFLISWRNPGPEQRDITWADYMQMGPISAMEAIEQACGEKSVNLLSYCVGGTLTASTVAYLTSTRRGRKVKSVTYMATLQDFRDPGDIGVFLNERVVEGIENTLEMKGYLDGRSMAYTFNLLRENDLFWSFYINNYLKGEIPAAFDLLYWNTDGTNLPAGTHAWYLRHMYLENRLVEPGGIELDEVKIDLRKISAPCYFVSTKEDHIAKWNSTYYGALLPKGPVTFVLGGSGHIAGIVNPPHKNKYGYWTNDALPETHDAWQEGSTFNEGSWWPHWQAWVTENGYADPDPEKMVSARQPGEGELNVIESAPGRYVKMTIPEVLGELPAS

SEQ ID NO: 24 - protein, 632 aa, synthetic construct
MMCLGRECQAGEGRVSMLSGWKMPSQGVSQEELEAWKVQLSDVGEQYKGLLEDLLSRIAPSEAADSVYSDMRESFEAAAQSLMSNPNLLWQTQSRLMQDQWLLWQQGVRTMSGEQVTPLITPAKGDRRFKDEAWTQEPYYLAIMQQYLLFSQMVEELIEDLDGLDPTQKRNLTFYARQLVNAMSPTNFVATNPEVMRCTLETRGQNLVDGLTRLREDLANSAEGINVRMTDRSAFGVGDNIAVTPGAVVYENELIQLIQYTPTTEKTFKTPLLIVPPWINKYYILDLREDNSLVKWMVDQGHTVFLISWRNPGPEQRDITWADYMQMGPISAMEAIEQACGEKSVNLLSYCVGGTLTASTVAYLTSTRRGRKVKSVTYMATLQDFRDPGDIGVFLNERVVEGIENTLEMKGYLDGRSMAYTFNLLRENDLFWSFYINNYLKGEVPAAFDLLYWNTDGTNLPAGTHAWYLRHMYLENRLVEPGGIELDEVKIDLRKISAPCYFVSTKEDHIAKWNSTYYGALLPKGPVTFVLGGSGHIAGIVNPPHKNKYGYWTNDALPETHDAWQEGSTFNEGSWWPHWQAWVTENGYADPDPEKMVPARQPGEGELNVIESAPGRYVKMTIPEVLGELPAS

SEQ ID NO: 25 - protein, 238 aa, synthetic construct
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 26 - protein, 225 aa, synthetic construct
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 27 - DNA, 2175 bp, synthetic construct
  source 1..2175: mol_type = other DNA
atgatctatcaaggcaatgccatcacggtggagcgtcgtgacacccaaggtggcaatgacatcgctaagctcactttcgatctgaaagatgagtccgttaataagctgtccagtgctgttgtggcagagttgggcgaggcagtaaaagcgcttcaagctgaaagcggcctacaggggttgatgattagcagtggcaaagatgcatttattgtcggcgctgacatcaccgagtttcacagcctttttgacaaaggcgaagagtatctcgtcgagatgaaccttaaggttcacgacatcttcaatgcgattgaagatctgcctttcccaaccgtgacggccattaatggcctggcccttggtggcggctgtgaagtcctgttgaccaccgatttccgggtaatgagtgagaaagccaaaatcggcttacctgaaaccaagctaggcattctgccaggctggggtggttgtgtacgtttacctcgcttgattggcgcagataacgcgattgaatggattgctggcggtactgaaaaccgtgctaatgcggcacttaaagtcggcgcagtcgatgctgttgttactcacgagcttcttgaagaagccgcattggatattctagaccgtgccaatgccggtgagttggattaccaagcgcgtcgcgaagagaagaaatcaccgcttaatctcaacgccattgagcaaatgatggcgtttgaaaccgccaaagggtttgtagctggcaaggccggccctcactaccccgcgccggtagagtcgatcaaggtgattcagaaaggcgctggcgagacgcgtgcccgtgcccaggcgattgaagcgaaagcatttgccaagctagcactcagcagtgttgcctttaacttggttggcctgttccttaacgaccaagtcgtgaagaaaaaaggtagcaagtacgagaagcagtcgcagccagttaagcagaccgccgtgctgggcgcaggcatcatgggtggtggtatcgcctaccagagtgccagcaaaggtacgcccattgttatgaaagatatcaatgaggaagccattgagctgggcctgaaagaagcgcgtaagctgttctccaagcaggtagagcgtaaaaagctgacgaccgagcagatggccgaaaagctgaccaacattcgccccacgctctcttacggtgatttcggcaacgtcgacctagtggtcgaggcggtggttgaaaatcccaaggtcaaagacgccgtactgaccgaagtcgaaggcatggtgagtgaaagcaccatccttacctctaacacctcgacgatttctattaatcgcttggctaagaatcttaagcgtcctgaaaacttctgcggtatgcacttttttaaccctgtgcaccgtatgccgttagtggaggttattcgtggcgagaagacctcagatgcggcggtggcagcaacagttgcctacgcacgtgcgatgggcaaaaccccgatcgttgtgaatgactgccccggctttttggttaaccgcgtattgttcccttacttcggcggctttagcttcctggtagagcagggcgctgatttccagcgcgttgacaaagtgatggaaaagtttggctggcctatggggcctgcgtatctgctggacgtagtcggtcttgatactgcggtacacgctaacgaagtgatggcagaaggcttcccggatcgcatggcgcgtgacggcaaaaccgctattcaggtgatgtacgacaataagcgtctgggccagaagaacgataaaggcttctatgcctatgaagaagataagaaaggcaaacctaagaaagttaccgacgagcaagcctacgcgctggtgaaagacgtcgtgaaagagcagaaagagttttctgatgaagacattatcgctcgcatgatggtaccgctttgtctggaaactgttcgttgccttgaagatggcatcgtggaaaccccagctgaagccgatatggcattaatctacggcatcggtttccctcccttccgcggtggcgcgctgcgctacattgacgcgatgggtgttgccgaattcgtgaagctagccgaaaaccttgcggaagagttaggcccgctttacgcgcccactgagaaactgcgtcagatggctcaaaataacgagcaattttattctggcactcaggcctaa

SEQ ID NO: 28 - DNA, 95 bp, synthetic construct
  source 1..95: mol_type = other DNA
aatagtggcgcggctaaactgttttagagctagaaatagcaagttaaaataaggctagtccgttatcaacttgaaaaagtggcaccgagtcggtg

SEQ ID NO: 29 - DNA, 95 bp, synthetic construct
  source 1..95: mol_type = other DNA
ttcacctagctagatgagacgttttagagctagaaatagcaagttaaaataaggctagtccgttatcaacttgaaaaagtggcaccgagtcggtg

SEQ ID NO: 30 - DNA, 95 bp, synthetic construct
  source 1..95: mol_type = other DNA
acaccattacgggggtgtcagttttagagctagaaatagcaagttaaaataaggctagtccgttatcaacttgaaaaagtggcaccgagtcggtg

SEQ ID NO: 31 - DNA, 95 bp, synthetic construct
  source 1..95: mol_type = other DNA
ttgggtgatatttagttgccgttttagagctagaaatagcaagttaaaataaggctagtccgttatcaacttgaaaaagtggcaccgagtcggtg

SEQ ID NO: 32 - DNA, 2893 bp, synthetic construct
  source 1..2893: mol_type = other DNA
ttgcgttcactggaatcccaatatagagtttgacctgcgagcaagctgtcaccggatgtgctttccggtctgatgagtccgtgaggacgaaacagcctctacaaataattttgtttaatactagagaaagaggagaaatactagatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcaacgccatggcccccagcaacttcctggccaccaaccccgagctgctcaagctgaccctggagtccgacggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgatgataagccaggcatcaaataaaacgaaaggctcagtcgaaagactgggcctttcgttttatctgttgtttgtcggtgaacgctctctactagagtcacactggctcaccttcgggtgggcctttctgcgtttatatactagagccgcttctagagctcggtaccaaattccagaaaagaggccgcgaaagcggccttttttcgttttggtcctactagatgcctccacaccgctcgtcacatcctgttgcgttcactggaatcccagactagagtttgacctgcgagcaagctgtcaccggatgtgctttccggtctgatgagtccgtgaggacgaaacagcctctacaaataattttgtttaatactagagaaagaggagaaatactagatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaactgctaacaaagcccgaaaggaagctgagttggctgctgccaccgctgagcaataactagcataaccccttggggcctctaaacgggtcttgaggggttttttgctgaaaggaggaactatatccggattactagaggtcatgcttgccatctgttttcttgcaagat

SEQ ID NO: 33 - DNA, 4658 bp, synthetic construct
  source 1..4658: mol_type = other DNA
aatagtggcgcggctaaactgttttagagctagaaatagcaagttaaaataaggctagtccgttatcaacttgaaaaagtggcaccgagtcggtgctttttttgaacccggggtaccggatgttgaggcgtttcagcaaaacctgctggaaaacattcagcggcaaatggaatctggggtggatggctatgaccccatgaatctaatgcgaccttttctgcagcagccaatgatgcagcagggagtctcgcaagggctggctaatttcggcacgtatcaacagatgatgttggatatgttgcgcaaagcgggaacctcaggcggcagtgctgaacaggatagcgacacgagccaagacgagtcccaagaagcagctaaaccaaagccagcagcatcaggtaaaagcgcatcaagtgcgagaagctccaaagcgaaagcgacagcttcttcgcgttctcgcgctaaagggtagaaatgtaatgtgaaatgtgtagcgttgttgagcgtgctgctaaggatgttgcaaagcagcattgcagcgtttactctttttgcagtgcagcaattttgccatgtgatgagccactccaggctcatgccaaggaacgagagacaggagcagacactatgcaagataaaatgatggacgcctttagtacccaaactcgccaaatgtttgagccaatgcgtaaaatgaactcgctaatgctcaacaacatggaaaaaatgactcagtatcagctggaagcgatgaaacgctacagccagatgggcactgagcgcatccgcagcgcgacagaaattaatgatgcagaaagcctgcgtgattttagcaccaaacaagccgaaatgatgaacgagctttctcagcagatgcaggaagatgctcgcgtcatgggtgagatgagtttgcagttcaaatccgaaatggaaaagctgttcagcgaagctggtcagaagatgggcgagcaagccacctctgccacgaaaagtgaacagcctgcgaaagcgactagccagtcttcacgtaaaagctaaacgcaactatgttcgcggcgccttgaaggcgccgcgttcttttggatgatgtgcctgggcagataatgccaagcaggtgagggagagtgagtcgttacctaccgcttctaacttgcgttcactggaatcccaatatagagtttgacctgcgagcaagctgtcaccggatgtgctttccggtctgatgagtccgtgaggacgaaacagcctctacaaataattttgtttaatactagagaaagaggagaaatactagatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcaacgccatggcccccagcaacttcctggccaccaaccccgagctgctcaagctgaccctggagtccgacggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgatgataagacaccgctcgtcacatcctgttgcgttcactggaatcccagactagagtttgacctgcgagcaagctgtcaccggatgtgctttccggtctgatgagtccgtgaggacgaaacagcctctacaaataattttgtttaatactagagaaagaggagaaatactagatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaactggacgctaaaactactaaccgagacgctactcatgcagccaactgcacgcttttcattgtcgccggctcttcctgagacccacactcaacacagccatggagcttgctgttgtggttcaccattgctgcagcgtttccatgaacgtgtgatggcagaaataacccggcgccaaatgattgggggaacggctgcggtgatggcgcttttcgcaggattgcatgtgccttttgctgttgggcagcagccaaaatcccacgatgggccgctactattaaccaatttaaagctttttgatggaagcggtggcccgctgcgtgatggcgtatcaatcagggtggtggacggccgtattgatgcgattctagacggcagtggtgaagtggaaggtattgatgtcattgattgcggcggtcgtgtattgatgcccggcttgattgatgcccactggcataccaccttagcggccatctcccagatggaagccatgaccgctgatattggctatgtgcatttggtcgcagcaagagaagcagaacgaaccttaatgcgtggcgttacctcggttagggatgtgggtgggccgtcgtttgcgttaaagcgtgctattgatgaaggtattgttaatggccctcgggtgtttcctgccggggcgatgatttcgcaaacctcgggtcacggcgacttcagaatgcgtcatgacataccccgtggtagcacaacgccgctaagtgaggcagagcgtcagggtgttgccgtgattgcggatggtgaagctgaagtgctcaggcgcacgcgtgagcagctgatgttaggtgcctctcagatcaagctgatggcgggcggtggtgtggcatcgctttacgatccattagacagtacccagtttacggaacgagagcttcgggcagcggttgaggctgctggtgactggggtacctatgtgatggtgcatgtctatacgcctcgtggtatccagcgcgctctgcgagcaggtgttaag

SEQ ID NO: 34 - DNA, 4645 bp, synthetic construct
  source 1..4645: mol_type = other DNA
ttcacctagctagatgagacgttttagagctagaaatagcaagttaaaataaggctagtccgttatcaacttgaaaaagtggcaccgagtcggtgctttttttgaacccggggtaccgtgcgcttgccacagcttcgcgctcgttctgtggctccggcagcatcgacgaatagtaggcgattcgctggaaacggtcggtcggcttgacgttccctggcagcggtgtatcgctgcttgggtttgaaaagtctaattcttccagcagtgccaattgctgatcgtaaggcggatcattggtcatgattctaaattctctaccgtggtgaatcatggattcaccatcgatatattcaatgatcgctgaatccccagtagcatcttcaattgccaaatgtacggttgccttactaccccgcgcctccgccatgacaatttgtacatcttcgagtatgtcgagtgcttcttcgacagtggctgcgttgtcgatcacatattgcccccacaaacctgcctgcattcctggtttgccagggtcacgcgggccaaagtcggtggccgtgagatagagcatgtggaacgccagccccttttcgttgaatccatcggcagcgccgatgccataaaccgtggtgaccatgctggcgtacttcgacgtccatgttgctgcgttctcctccacgacgacttccgcgcccagcatgcccccatttcgctccatgccacgggggaagacagtaataaccggatctgtcgattcgggccagtccattgtgcgggcaacataaatgccctgctcgttgtcattccataagtacctagaacacgcccatgctgcattggccattaccatcgcaccaatcacgatggcaacgccccctctgtaatacctacgcatgaattactcctttagagcgtgtaaatcgccagcacaacgcccttatgaaaagacgcttaaatcgagcgtctgattaaaggccgatagcccaaagcaggctcgagcaatcagccttccacccaagctattttcgttaattctttaccccagaaaaactccacaaatacaaaatatagttaacttttgaggcgatgcaaatccaccttccgagcacatcgatcacgttacctaccgcttctaacttgcgttcactggaatcccaatatagagtttgacctgcgagcaagctgtcaccggatgtgctttccggtctgatgagtccgtgaggacgaaacagcctctacaaataattttgtttaatactagagaaagaggagaaatactagatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcaacgccatggcccccagcaacttcctggccaccaaccccgagctgctcaagctgaccctggagtccgacggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgatgataagacaccgctcgtcacatcctgttgcgttcactggaatcccagactagagtttgacctgcgagcaagctgtcaccggatgtgctttccggtctgatgagtccgtgaggacgaaacagcctctacaaataattttgtttaatactagagaaagaggagaaatactagatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaactgctcaccttcatttccttatgctgaacacggtaagcttggcgataacaccgctactgatggctaaaaatcaacgatgacttcccattggatacttggccctggggccattggtcgcctgttggcgcattcgctttcacccattactgacatcaccttgattggccgacgtgcattgcctgaacagcaacgcctgaccacccctgaaggtgaagaacgaactcagcggctagcgagcgttacggttgcggagctagcctctcactcactgcctgtgcctgggtttgtgcatatcaccaccaaagccatggccgccgaggcggcgcttgccagcattgccgatgtggttgcgcccacgaccccgctggtgctgtggcagaacggttttttggcacagccacggctgactgataggtggccagggccagtgctgtgtgcgaccacgactcaaggcgcttacttaaccggcagtgatggcgtggttcacgccgggcgcgggccgacgtttattggtgatctcaacaatcaacgcgctgcgctagcaaaaacactggcgcagacgttaggtgaggcaggctttaccgccacgccggtagacgatattcgccaacgcctgtggcaaaagctggcggttaatgcggcgatcaacccattggtggcgctcaatggcgtgcgcaatggtgagctgcgcggtgatgcttatgcagtccgtttggcagcggtggtaaaggaagtcgcggcgattttaaagcaggaaaacattgcaccaccgaatggtggcgaaggtgaagacgcgtggttggcgcttgtgtggcaggtggtggagaacaccgctaacaataaggcctcgatgttgcaggatgttgaggccagacgcaccaccgagcgcggggcgattttagggccgttgattgatagcgccgagcgccatgggttgccgtgtgggttgttgaaggagcttgatagcgaattggctaaattggaggcgaagttttagggaaca

SEQ ID NO: 35 - DNA, 4643 bp, synthetic construct
  source 1..4643: mol_type = other DNA
acaccattacgggggtgtcagttttagagctagaaatagcaagttaaaataaggctagtccgttatcaacttgaaaaagtggcaccgagtcggtgctttttttgaacccgggatgtatcaagctgcccgttgctgaccagatttctgtctgatactagatttttggccagattctggtgtaacgcctgattgaccgaagcgttgtattcacgcagcgaatagaggctgatgaaggtatatagcaggcccacggcgagcaataacagaaataagcctaaggctagccgagtatagagtgttttcagcataacttattcgcggaagcgatagcccacccccccaacggtttggataaacaccgggtcagcggggtccgcttcgatcttgcctcgtaagcgattgatatgagtattgacggtatgttcgtaaccttcgtggctgtagccccacacggtatcaagcagttgggcacggctaaatacccgccctggatgtgtggcaaagtgccatagcaggtcaaactcccgggcagtgagttcaacaggctggtcttttataaatacccgccgacgcagcgggtcaatgcgcagcccatcggtgattaatgcttgctggctggggtccgtgactgaggaagatgccatggcatctacgcgtcggaacaaggctttcacccttgctgatagttcagccacactgaaaggtttggtgaggtaatcatccgcccccatttccaggcccagcactcgatcaagctccgtacttttggcggttagcatcagtaccggaacgtaacctgggccggcgcggatttcgcggcaaattgacagcccgtccaggccgggcagcatcaggtcgagaaccacaaggtcgatgcccccttcacgaaaacgctcaagcccggaatcaccgcgctcgcaaagaatagggttcatgccaagctcggccacatgcatgcggacgagttcaccaatacccgggttatcctcaataatcagtacgtttcgtgtcataggtagtcgcattatcttcttttgtgctttcgccttatggctctaggcgcttggatgctcgtgatattttcctactcattggtcggaagattcgagactatccgttacctaccgcttctaacttgcgttcactggaatcccaatatagagtttgacctgcgagcaagctgtcaccggatgtgctttccggtctgatgagtccgtgaggacgaaacagcctctacaaataattttgtttaatactagagaaagaggagaaatactagatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcaacgccatggcccccagcaacttcctggccaccaaccccgagctgctcaagctgaccctggagtccgacggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgatgataagacaccgctcgtcacatcctgttgcgttcactggaatcccagactagagtttgacctgcgagcaagctgtcaccggatgtgctttccggtctgatgagtccgtgaggacgaaacagcctctacaaataattttgtttaatactagagaaagaggagaaatactagatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaactgggtgatatagagtgtatcgcgcaaagttcacaaatgtgtcacggggactctaacgatagggcgatgtctgcattaacgcctcccctcatttcaataagtgtgtttcagcaaggagagcgcccatggcgactacacgccgtaaactcatgtcgttaatcgggcagggagtgatacctcctgagcaagtgccgttagcggttaaagtggcggggctgcacccgtcgccccacgcctgggcgctgttgattgaccggctgctactgtggctggggagcttggcgttggcttgcgcggtgctgttcttcgtggctttcaattggtcggacatgggccgcctgccgcgctttgcgttagtgcaggctgcgttggtgttggcggcaggcatcgcggtgtggggcagcgcgagcgtgatgctttttcgcgtgacgttaactgctgcgtctctattgataggagtactactggcgctggtgggtcagatttaccaaaccggtgctgacccatggcagctgtttttcacctgggcgctgctcaccttgccattggtatggatagcacgctttgatgcgctttggttggcgtggttagggcttttgaacctatccgtgtggctgtatagcagcacctggggcggtgtttttggcaatgtgttctctgccgataacgcgggcttatggggactggtgctgatcaacttagtggctcaagtcgtgtgggagtggggcgcgcagcgccgaggttggccggggcgttgggcgatttgcttactggcgctaggtagcggcgtgccactgacgctactgatgatggcgtgggtgagcggggaaacgcatgcgttgacaccgattgtggcggtttaccctgtgtggttggcagtgctgtatggcgtttatcgtcagtggcggcttgagctatttatgttggcagggggctgcgtctcggtgattgcggtggtcacattgctgcttgcacgctacatgctatgggaaagccaatggaa

SEQ ID NO: 36 - DNA, 4785 bp, synthetic construct
  source 1..4785: mol_type = other DNA
ttgggtgatatttagttgccgttttagagctagaaatagcaagttaaaataaggctagtccgttatcaacttgaaaaagtggcaccgagtcggtgctttttttgaacccggggtaccgcttatcggcaagcggatcactctggagcagtcgattgatgcgctgatgagcatggataagttcgaaggggcgggcgtaacggtgggaccgagttttgagataacgggcttcagcatcactgcccgttctaaattgaaaggcttaggtaagactgacgctaatagcatcgagggcgttgaattagcatctactcacgatgtgtggtgtgcggtaggctcactaattgcttccaacctgattggacaccctcatgctcaccttgcgcactgtcactttcacgcttctggtcgttattctcggcatactgcccatcaacgcttacgcaaacgaatcaacttggcaggcgctgcaggaaggcggcttggtgattctgatgcgtcacgcactggctccaggcattggtgatccggctgggtttgagctttctcagtgcgatacacagcgtaacctttcagcccaggggcgcgctcaggctgaggcgattggtcgttcatttcgtgagcgggatgtgcctattgcctcggtgtattcgtctcgctggtgccgggcgctggaaactgctgagctaatggcgttgagccgcgtggagccaaccccatggttagattcgttttttcgtcagcgcagtgagcgaggtgcacgcacgcaaacggcgcgtgagcacatagctgcttggcatggcccaggtaatttgctgctggtgacccatcaggtcaatattacggcgctcacaggcggtggagtgagctcaggcgaaatgatcgtcgtgcgcccaacgggcgattcgtttcagatggtgggccggttgaacgatgagtaggcgatgaccgctaatcgctagcgggcgttttaagtagcattagtgcttccttaatggtttttattagtcaattaaaaatcaaagtgttaaagaaacactttttagcagtttaattaattcatgatttttagcgttaattattaatgcgataattgtttcttattgtttaatttaaagccaaggcgttacctaccgcttctaacttgcgttcactggaatcccaatatagagtttgacctgcgagcaagctgtcaccggatgtgctttccggtctgatgagtccgtgaggacgaaacagcctctacaaataattttgtttaatactagagaaagaggagaaatactagatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcaacgccatggcccccagcaacttcctggccaccaaccccgagctgctcaagctgaccctggagtccgacggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgatgataagacaccgctcgtcacatcctgttgcgttcactggaatcccagactagagtttgacctgcgagcaagctgtcaccggatgtgctttccggtctgatgagtccgtgaggacgaaacagcctctacaaataattttgtttaatactagagaaagaggagaaatactagatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaactgtgataagccaggcatcaaataaaacgaaaggctcagtcgaaagactgggcctttcgttttatctgttgtttgtcggtgaacgctctctactagagtcacactggctcaccttcgggtgggcctttctgcgtttatattttaaaggctggtatataaaagcttgtctttgttgtaggcgattagttgaatgagtttttacactgagtcgctatgacagaatttaacaatgtggttagtttttaagaactagtttcttacttttgattaattaaacaagagtcgtgcactgagtgcgcgcattactgcgtacacatgaataagtgccttgtcggtgcttaattagctggcgcggtgttcccaatggtgcttggacattagaggagtagggatatgaaaaagtacgcagcggagttaatcggtacgttttggttagtgttgggtggttgtggcagcgcggtgctagcggcggctttcccagacgtaggtattggcctattaggggtatcgctggcctttggtttaacggttgtcacgatggcgtatgcgattgggcatatttcgggctgccatttaaaccctgcggtatcgatagggctttgggtgggcgggcgcttccctgcaaaagagttgccttactatattggcgcgcaagtgctgggtgctattgttgccggcggtgtgctgtatcttattgcgagtggtcaggcgggttttgatgtgtcggctggatttgcatccaatgggtatggagagcattcacccggcggttacagcatgatggcagcactactcattgaagtcgtgatgaccatgatgtttatcttcatcattatgggggcaaccgatggtcgtgcaccggcaggctttgcgcctttggcgattggcttgggcttaacgcttattcacctgatcagcattccggtgaccaatacctcggtgaatccagcgcgcagtacgggcgttgcgctgtatgtgggtgattgggcgacggctcagctgtggttgttctgggtagcgccaatgatcggtgcggcgttaggggcattagcgtatcgctttattgctcagcctgaggcactcatagccccgccatcgccttctgatcacgatgatgatctgacgggtaagatcgttcc

SEQ ID NO: 37 - DNA, 96 bp, synthetic construct
  source 1..96: mol_type = other DNA
attcgccccacgctctcttagttttagagctagaaatagcaagttaaaataaggctagtccgttatcaacttgaaaaagtggcaccgagtcggtgc

SEQ ID NO: 38 - protein, 724 aa, synthetic construct
MIYQGNAITVERRDTQGGNDIAKLTFDLKDESVNKLSSAVVAELGEAVKALQAESGLQGLMISSGKDAFIVGADITEFHSLFDKGEEYLVEMNLKVHDIFNAIEDLPFPTVTAINGLALGGGCEVLLTTDFRVMSEKAKIGLPETKLGILPGWGGCVRLPRLIGADNAIEWIAGGTENRANAALKVGAVDAVVTHELLEEAALDILDRANAGELDYQARREEKKSPLNLNAIEQMMAFETAKGFVAGKAGPHYPAPVESIKVIQKGAGETRARAQAIEAKAFAKLALSSVAFNLVGLFLNDQVVKKKGSKYEKQSQPVKQTAVLGAGIMGGGIAYQSASKGTPIVMKDINEEAIELGLKEARKLFSKQVERKKLTTEQMAEKLTNIRPTLSYGDFGNVDLVVEAVVENPKVKDAVLTEVEGMVSESTILTSNTSTISINRLAKNLKRPENFCGMHFFNPVHRMPLVEVIRGEKTSDAAVAATVAYARAMGKTPIVVNDCPGFLVNRVLFPYFGGFSFLVEQGADFQRVDKVMEKFGWPMGPAYLLDVVGLDTAVHANEVMAEGFPDRMARDGKTAIQVMYDNKRLGQKNDKGFYAYEEDKKGKPKKVTDEQAYALVKDVVKEQKEFSDEDIIARMMVPLCLETVRCLEDGIVETPAEADMALIYGIGFPPFRGGALRYIDAMGVAEFVKLAENLAEELGPLYAPTEKLRQMAQNNEQFYSGTQA

SEQ ID NO: 39 - DNA, 693 bp, synthetic construct
  source 1..693: mol_type = other DNA
atgttgtctgcgttgcagcacgagttagacgctatcgctgtcgaccctgctgcgcgctgcgtagtgattggcagtaatgggcgtgggttttgtgctggtcatgatctgaaggaaatgcgctcgaatcctgaacaagcctactaccaaacgttatttcgtaactgtagccgcttgatgcagagcatcgtcaaactgccggtgccagtgatcgccagagtgcaaggtatggccacggcggcggggtgtcagttagtggccagctgcgatctggccattgctggccgttccgcaagttttgcggtgtcaggtatcaatgttggactgttctgctcaacccctgcagtggcgttatcgcgctgtgttgcccccaaacaggcattcgatatgctgatatcaggcgagtttattgatgcagagacggcgatggcgtgggggctggtgagtgatgtcagtgaagacgctgacctggacgtggcggtggccgataaggtggcgcagattctgagcaagagtccggcggcggtgcgttatggcaagtctatgttccatgctcagcgtcagatgagtctggctgatgcctatgactttgccggtaatgtaatggccgaaaacatgcttaacccagatgcccaaaacggagtagacgccttcttagacaagcgtaaacccatttgggaacaacctggccaatga

SEQ ID NO: 40 - DNA, 774 bp, synthetic construct
  source 1..774: mol_type = other DNA
atgagcgatgcagtgattgaaaaagttgataacgacggcatggtgcgattgacgataaatcgccctaaagcattgaatgcattgaatagcgatgtgctaagtgccttagaagaacaccttgtggaacttgaggcgcacccacgtttacgcgctgtcatcatcactggcgccggtgaaaagtcgtttgttgccggtgctgatatcaccgaaatgcgtgataaaaccccagaagaagcgcgtgcttttgctacccaggcactgcggactatcaagcgcttagaaacattgcccgtgccggtggtcgcgctagtgaacgggttttgccttggcggcggctgcgaattggcactggcctgcgactgggcggtagccagtgaaaacgctgtcttcggccagcctgaagtgttgttaggcgtcattcctggctttggcggcacccagcgtttgccgcgtcgtgttggtcccgccatggcgattgacctagtaaccactggccgcaaaattgatgcccaggaagcgctgcgcattgggctagttaaccgtgtgatgccccaggctgagcttgaaaactatgtggaagagctcaccaagcagcttaaaggtaacggcccactgtctgtgcgcggtgctaagcaagccgttcacgacgggctagatcaagaccttggtagcgctctggcgctggaaaccagcctgtttgcgttctgctttgctggcgaagagcagaaagagggcatgagtgctttcgttgagaagcgcaaacctaacttctaa

SEQ ID NO: 41 - DNA, 1116 bp, synthetic construct
  source 1..1116: mol_type = other DNA
atgagtagcgtactgtttgctgaacacgccacccaagatggccatgttattggcgagatcacgcttaacgccgagcgttcactgaatgccctgacgttggagatgattgaagagattctgccgcgcttgaccgcgtggcaaaccgacgagcgagtcgttgctgttctgttggatagcgctggcgaaaaagcgttttgcgcagggggcgatgtcgtcaatttgtataaagcgatcaaaggagacggcgagccagatttcccagagcgtttttttgaacacgagtaccgcctggactacctgctgcacacctatcccaagcctgttatttgctggggcagcggtattgtaatgggcggaggtatggggctacttagcggtagcagccaccgaatcgtaacggaaacctcacgtctggcgatgccagaagtaacgattggcctttacccagacgttggcgctagctggtttttaaaccgcctgccgaatggcaccggacgctttctagccatgaccggcagccaaattaacgcgccagatgctgttcatctgggccttgcggatcgcgcgattgctagccattaccgcgcagctctggctcaacaactgatggacgcaacctggggtgaaggaaatcaaactgacgcgcatggtgtggttaacagtgtgctgcgcgaatttgaacaagcctctcaagaggtatttgccgagctggaatcgccggttcacaaatccgccgcattgattcgtgagctaatggatcacgacacggttgagcaagccgtggatgccgtgctggcagtgagcagcgacgacaaatggttcagcaaagcgcagaaaacgctttcccacggtagccctgtgtcagtgaagctgattgatgagcagctaaagcgcgccaagcatatgtcgttggctgaggtgtttcagtttgagctggcgctttccgtgcagtgctgccgtcatcaggaattccccgaaggcgtgcgtgcgctgttggtcgataaagatggccagcctgcgtggaaatatccagatgtcgcgtcagtcgagcaagcctttatcgatgagttgctggcttcaccgtgggaaaagagccccctagcagatcttcgctaa

SEQ ID NO: 42 - DNA, 834 bp, synthetic construct
  source 1..834: mol_type = other DNA
atggcttactcaacgctattgatcgaaaagggcgtggcgcgcctgactttaaaccgcccagaggtgcataacgcctttgacgacagcctgatcgctgaactgaacgcccatctagacgaattgcatgcgctagccaacgccggcgacgttcgggtagtggtacttggctccgaaggtaaaagcttctctgcaggcgctgaccttaactggatgaagcgcatggtcgattacgacctggaaaataacttagcggactctcgtaagctctcagcactaatgtatgggctagataccttgccctgccccacggtttgccgtgtgcagggggccgcctttggcggcgcggtgggtcttgccgcctgttgcgacctggtgatcgcttcagataaagctaaattttgcctgtcggaagtgaaaattggcttatcgcctgcggtgattagcccctatgtgcagcgcgcgcttggcgagcgccaaatgcgtcgttatgcactcactgctgaagtgatggatgcaccgacagcgatggcactcggtttagcccaccaagtcgttgaacacgatgccttggatggcgccgtagacgctatgctcgacacccttctgggtggctctccccaagcccaacgggcaaccaaagcactgctggcaagcgtggctcaggcacccgacagtgacgccacccgagaacacacctgccaagtgatctccaaactgcgggtgagccaagagggtcaagagggcttagctagcttttttgaaaagcgccgccctgcctggacgcaaccgtctggccttcaagaacccaacactgctgcggagccacgctcatga

SEQ ID NO: 43 - DNA, 792 bp, synthetic construct
  source 1..792: mol_type = other DNA
atgcatcaaatcctcaatgaacgtgtaacgctgaccttcgccgaccacgtggccgaagtcatgttgtcgcgacccgaacatcacaatggcctggattgggccatgatcgatggcctgctggcggcccagcagcgcttgacccagcttagtggagtgcgtgccgtggtcttaagcggcgatggtgacagcttttgcgcggggctcgatatggcggcgatcatgatcgaggcggagcgattgcccactctgctggcgcctgacgaagcgggcatcaacccggttcagcgcttggctctaggctggcgtgaggtgggcgttcccgtggtggctgccttgcacgggcatgtctacggtggcggcctgcaaattgccttgggtgccgatatccgtattctccatccacgagcacgactggcgctgctggagatcgattggggcatcatccccgatatggctattagcgtgactggagcggcgttgcgccaggatgtgctccaagaactggcgatcagcggacgcaaggtgtcaggggaagaggcctatcagttaggcctgggcagtcggctgagcgatacgcccctggaagccgcccgggaaaccgctgcactcatcgcctcgcgttcacctcgggccgtcgctgcggctcgtgagctgttcgctcgcggtccggggctggcccatggcgatcgtctggcccttgaagcgcatttgcaacagcagcttttggccggcgccgagcatcgcgaagcggtgagcgcacgaatggagcgtcgcgccccgcgtttcgactga

SEQ ID NO: 44 - DNA, 792 bp, synthetic construct
  source 1..792: mol_type = other DNA
atgagccaagcgcccctgctttacaccgtggaagatggcgtcgcctgtatcacgctgaaccgccccgacagcctgaacagcttcaatactgaaatgcatgctgaaatgcgcaaagcacttaaggccgtgcgccaggactcaagcgttcgcgctttgttactgaccggcaacggccgcggcttctgcgccggtcaggatctttccgaccgcagcgtcgcgcccggcgagcaagcccccgacctcggcgagtcgcttgaaaaacgctacaaccctatgctgcgcgcccttaaagatatgccgttcccgactatttgtgcggtgaacggcgtggccgcaggtgccggcgccaacatcgccctggcctgcgacatcgtattggctgctcgttcggcaagcttcatccaggccttctgcaagattggattaattccagactccggcggcacctggacgctgcctaacttggtgggtatggcgcgcgccaaggggctggcaatgcttggcgacaagcttcccgccgagaccgccgaacagtggggcatgatctggcgctgcgtcgatgatgaggcgcttcaggaagaagccatgaacatggctcgccacctagccactcaaccgacgcgcggcttggcactgatcaaacgcgccctgcatgccagcagcgataacagcttcaacgagcaactcgacctggaacgcgacctgcagcgccttgccgggcgcactgaagactaccgcgaaggcgtcagcgcttttatcgagaaacgtcgccccaccttcaagggagagtga

SEQ ID NO: 45 - DNA, 807 bp, synthetic construct
  source 1..807: mol_type = other DNA
atgtctgcctctccagatgcgccactggcgtcttcagatctgctgcaacgcactgactatcaaggggtaacgacccttactttaaaccaacctgaacgctttaatgcgctgtctgaggacatgctgacggcgctgcaacgtgcgcttgatgatattgctcagaatgagcaagtgcgctgcgtagtgttagccgcgaacggcaaggcattctgcgcaggacatgatttaaagcagatgcgtgcgaaccctgataaagcctactaccaagcgctgtttgctcgctgcggcgcggtaatgcagaccatcgttaacttgcctgtacccgtgattgctaaagttcagggcattgcgacggccgctggctgccagctcgtcgctagctgtgatttggccgtggcggctagcagtgcccgctttgccgtgtcagggatcaatgtggggcttttctgttcgacaccggcggtagcgctgtcgcgtaacgtatcgcgtaaacgggcgatggaaatgctgctgaccggggagtttattagtgcttctcaggctgccgactgggggctcattaatcgggttgccgaagagagcgaactcgatagtactgtggaagcgctcactgcaagcatttgcgctaagagcgcggtggcggtgcgcactgggaaggcaatgttcgcccgtcagttggccatgccgttagaggaagcctatgcctttgctggtgaaaccatggcgtgcaacatgctggcagacgatgtagccgagggcattgatgccttcattgccaagcgcccgcctcagtggcgccatcgctag

SEQ ID NO: 46 - DNA, 849 bp, synthetic construct
  source 1..849: mol_type = other DNA
atgagtgaacaaccaatcggggtagtgggagctggcaccatggggcaggggatcgcccaggtggtcgctgccagtggttttacggttcgtctctttgatgtcgccgatgagcagcttactcgtgcccaagccaatattgataaaggtcttggcaagctggtcgcgaaagaaaaaatgagcgagtcagctaagcaggaggcactgtcgcggctttccactactactgatctagaagcgctaagtgactgtggcattattattgaagccgccccagagcagcccgcgctgaaagagaagctgtttcgtgaccttagtcatttaagcagcgacgcgattcttgcctctaacacctcgtcactctcgctaacgcggctagccgctgtgtgcgagcgacccgagcgggtagtgggcatgcactttttcaacccagtgccggtgctcaagttagtggaagtgattcgtgctgaacaaacctctgataccaccgttgagcgcatagaagcgctggcgaaagcgcttggtaaaacggcggtgcccattggtgatgcaccgggctttgcggttaatcgcttgttggtgccgatgatcaacgaagctgcctttatcgttcaagaaggaaccgcaacgccggaggccattgatgaagcaatgaagttgggcgctgcccacccaatggggccgttggcgcttgcagacttgattggtctcgatgtttgtttggcgatcatgaatgtgttgcaggaagggtttggtgaccctaaatatcgcccctgcccgttgctaaaacgcatggtggatgcggggtacctgggacgtaagagcggtcgcggcttccatatctacgagtga

SEQ ID NO: 47 - DNA, 957 bp, synthetic construct
  source 1..957: mol_type = other DNA
atgagccgccaacttagcgtgataggaaccggcgtcattggcaatggctggattgctcgcgccttggcacaaggctgggatgtcgtggcctttgacccagaccccaatgcacccgaacgcacgcgtgcctttattgacaatgcctggccctctttacaacgtcttggcttggcagaaggcgctaacccaaaacgcttacggttcgtcgatagcgtagaagaggcggttattggcgctgacttaattcaggaaaacgtacctgagcgacttgagcttaagcgcgaaatgcttgcggcgctggattcagctgcggaaaagcacgtcattatcggctcttccacatccggctataaacccagcgacttgcagcaagactgccacaatgctcctgggcgagttatcgtggctcatccctttaatccggtctatctgctaccgctagtggagctcgttggcggtgaagcgaccgcgcctgacgagttagcacgcgcccacgatcactatcaagcacttgccatgcgaccactggtggtgcgccgggaaattgaggggcatattgcagaccgcctgatggaagcgctttggcgcgaggcgctgcacctagtcaacgacggcgtcgccactaccgaagagatagacgcggcggtggtctatggctgtggccttcgctggtcactgatgggcactttcctcaccttccatttggctggcggcgagccgggcatgcgccatatgttggagcaattcggtccggcgctaaaactaccgtggacaaagcttgaagctcctgagcttaccaacgaacttattgatcgtgtggtggcaggctgtgaacaccaggccgcaggacgctcggtggccgagcttgacagccgccgtgatgactttctggtggagctactcggcctagtacaaaaatattggcctgaagccgaaggcttggaaggacgtatctga

SEQ ID NO: 48 - DNA, 1539 bp, synthetic construct
  source 1..1539: mol_type = other DNA
atgccagcccttcccacgtctgccgttattggtgtgattggcgcaggtgccatgggtgccggtattgctcaggttgctgcccaggctggccactcggtggtcttgcacgacaatcagccgggtgccgccaaagcaggcatcgataacattcgccgccagctggaaaagcgcgtcgccaagggcaagatgacccaaggcgatgtggataacatcgtcgctcgcctgcagcccgccgatgctatcgatgccttggccgacagccgcttggttatcgaagctatcgtcgagaaccttgagatcaaacgtcaggtgttcgcgcagctcgaagcgttatgctcaccggataccttgctcgccagcaatacctcgtcactatcgatcacctccattgcagcgaaccttgaacgccctgaacgcatggcggggctgcacttcttcaaccccgcccccgtaatgaagctggtagaagtggtgtcgggtttagcgaccaccaccgacgttgccgaaaccctttacgccactgctagcgcctggggcaaggtcgcggtacacgccagctccacccctgggttcattgttaaccgcgtggcacggccattctatgccgaagggctacgactgctgcaggaaggtgccagcgatgccgccacgatagatgcgctgctgcgtcaggcgggtggcttccgtatggggcctttcgagctaatggatctgattggtcatgacgttaactacgcggtgacccgctcggtgttcgatgcctactacggcgatacccgtttcgagccttccttagtacagcaagcgctggtggaagccggacgcctggggcgtaagtccggccaaggcttttttgactacgcgggtgaaaacactgcgaagccacagcctaagtttgccgcaccagcaaacgcggaactaccagcacttgtcgtacagggcgaccctggtgtgatcgctccgctgctggaacgcgcccaggaagctgggctcgacgtagctcgtcgtgaatcactccagtcagacggcacgcctcgcttatatctgggcgatctggtgctggcccttagcgacgggcggtgcgccgccgagcgcgcggtcaatgaagggctcgacgccctggtcctgttcgacctgtgcctggactaccgcagcgccagcgctatcgcgcttgctgccagccacatgacatcgcccgaagctcgtcaactggcgacggcgttcttccaacgcctgggggtggacgtgacttggctaaccgacaccccggggctggtggtactgcgcaccgtggctatgctcgccaatgaagctgccgacgccgtactgcaaggtgtttgcagcgcccaggatggcgacctggccatgcaggctggcgtcaactatccccgcggccccttggcctgggcagactcactgggcctgcctttcgttcaccgtaccctgacgcatctacagcagagctacggcgaagagcgctatcgcagctcgtttctgctgcggcgcaacgccttgagcgaggaaagctttcatgagtga

(19) United States
(12) Patent Application Publication
(10) Pub. No.: US 2026/0250692 A1
(43) Pub. Date: Aug. 27, 2026

(54) METHOD FOR PRODUCING 3-HYDROXYBUTYRIC ACID/3-HYDROXYHEXANOIC ACID COPOLYMER PHBHHx

(71) Applicants: TSINGHUA UNIVERSITY, Beijing (CN); BEIJING PHABUILDER BIOTECHNOLOGY CO., LTD., Beijing (CN)

(72) Inventors: Guoqiang CHEN, Beijing (CN); Huan WANG, Beijing (CN); Fuqing WU, Beijing (CN); Xu LIU, Beijing (CN); Wuzhe HUANG, Beijing (CN)

(21) Appl. No.: 18/726,070

(22) PCT Filed: Feb. 2, 2024
(86) PCT No.: PCT/CN2024/075554

§ 371 Date: Jul. 1, 2024

(30) Foreign Application Priority Data
Feb. 6, 2023 (CN) 202310137843.5

(51) Int. Cl. C12N 15/74; C08G 63/06; C12N 9/10; C12N 9/22; C12N 9/88; C12N 15/11; C12N 15/90; C12P 7/625

(52) U.S. Cl. CPC C12N 15/74; C08G 63/06; C12N 9/1029; C12N 9/226; C12N 9/88; C12N 15/11; C12N 15/902; C12P 7/625; C12Y 402/01119; C12N 2310/20

(57) ABSTRACT

The present invention provides a method for producing a 3-hydroxybutyric acid/3-hydroxyhexanoic acid copolymer PHBHHx. The method implements the production of PHBHHx by the heterologous expression of PHA synthetase PhaC and an enoyl-coenzyme A hydratase PhaJ, and achieves the purpose of controlling the molar proportion of 3HHx in PHBHHx by knocking out or knocking down the coding gene of endogenous enoyl-coenzyme A hydratase FadB in a halophile and/or adjusting the composition of a related carbon source, thereby regulating the material performance of PHBHHx.

### TECHNICAL FIELD

The present invention relates to the fields of microbial metabolic engineering, fermentation engineering, and synthetic biology, and in particular, to a method for producing a 3-hydroxybutyric acid/3-hydroxyhexanoic acid copolymer PHBHHx.

### BACKGROUND

Polyhydroxyalkanoates (PHAs) are environment-friendly biological polyesters that can be produced by the fermentation of a variety of microbes, and are among the prospective substitutes of petroleum-based materials. Meanwhile, PHAs have various application prospects in the fields of medical care, degradable materials, packaging and coatings, animal feeds, and the like. Currently, more than 160 monomers for forming PHAs are present, which endows PHA materials with great performance differences while meeting different application scenes. Poly(3-hydroxybutyrate-co-3-hydroxyhexanoate) (P(3HB-co-3HHx) or PHBHHx) is a PHA formed by polymerization of a short-chain (C4) monomer and a medium/long-chain (C6) monomer, and is one of PHAs with mature mass production and industrial applications at present, possessing great commercial prospects.

Traditional fermentation industry techniques have the defects of complex sterilization process, large fresh water consumption, susceptibility to bacterial contamination, and the like, which seriously restrict the rapid development of modern industrial biotechnology. In order to solve the problems, the Next Generation Industrial Biotechnology (NGIB) developed based on chassis extremophiles can achieve open continuous fermentation without excessive energy consumption, featuring ease of engineering and significantly improved robustness of the fermentation process. Halomonas bluephagenesis, a halophile isolated from Ayding Lake, Xinjiang by the Chen Guoqiang team of Tsinghua University, has the characteristics of wide environmental adaptability, salt resistance, alkali resistance, and the like, and is one of the important chassis strains for NGIB. The wild-type H. bluephagenesis can accumulate over 80% of PHAs without sterilization using glucose as the sole carbon source.

Therefore, in the face of increasing market demands, the development of a cost-efficient, sustainable, and efficient method for producing PHBHHx based on a halophile chassis and NGIB process possesses a very important promotion effect on further improving the cost-efficiency and overall benefits.

### SUMMARY

For the efficient production of PHBHHx via recombination halophiles, the present invention combines a number of metabolic engineering strategies, and achieves the systematic, iterative engineering process of halophiles from synthesizing PHBHHx to efficiently synthesizing PHBHHx, and further to synthesizing PHBHHx independent of antibiotics, and 3HHx-monomer-proportion-adjustable PHBHHx. By optimizing the gene expression level, the carbon source conversion rate is improved, and the cost-efficiency is further improved without using antibiotics and other means; the production of PHBHHx containing different proportions of 3HHx monomer can be customized by adjusting the carbon source proportion, so as to further meet more application requirements of diversified materials. The implementation of the engineering strategy has great practical significance in the aspects of improving PHBHHx product competitiveness, the benefit of mass production, and the like.

In a first aspect of the present invention, a recombinant halophile is provided. Preferably, the recombinant halophile expresses an exogenous phaC gene and/or phaJ gene, and/or, the recombinant halophile does not express a key protein of the β-oxidation cycle pathway or expresses proteins with no functions or reduced functions. Preferably, the recombinant halophile further expresses an additional polymerase capable of polymerizing 3-hydroxybutyric acid and 3-hydroxyhexanoic acid. Preferably, the phaC gene and/or phaJ gene are derived from an Aeromonas.

More preferably, the phaC gene or phaJ gene is each independently derived from Aeromonas caviae or Aeromonas hydriphila.

Preferably, the exogenous phaC gene and/or phaJ gene are each independently derived from Aeromonas caviae FA440 or Aeromonas hydriphila 4AK4.

In one specific embodiment of the present invention, the exogenous phaC gene and phaJ gene are both derived from Aeromonas caviae FA440.

In one specific embodiment of the present invention, the exogenous phaC gene and phaJ gene are both derived from Aeromonas hydriphila 4AK4.

In one specific embodiment of the present invention, the exogenous phaC gene is derived from Aeromonas hydriphila 4AK4, the exogenous phaJ gene is derived from Aeromonas caviae FA440.

In one specific embodiment of the present invention, the exogenous phaC gene is derived from Aeromonas caviae FA440, the exogenous phaJ gene is derived from Aeromonas hydriphila 4AK4.

Preferably, the phaC gene and/or phaJ gene are regulated by an inducible promoter and/or a constitutive promoter.

Preferably, the constitutive promoter includes, but is not limited to, a wild-type Pₚₒᵣᵢₙ or a mutant thereof.

More preferably, the Pₚₒᵣᵢₙ mutant includes, but is not limited to, one or two or more of mutant Pₚₒᵣᵢₙ₅₈, mutant Pₚₒᵣᵢₙ₄₂, mutant Pₚₒᵣᵢₙ₆₈, mutant Pₚₒᵣᵢₙ₂₇₈, mutant Pₚₒᵣᵢₙ₁₉₄, mutant Pₚₒᵣᵢₙ₂₂₁, or mutant Pₚₒᵣᵢₙ₂₀₃.

Preferably, the inducible promoter includes, but is not limited to, a Pₗᵤₓ promoter and/or a P_{lac} promoter.

Preferably, the phaC gene and/or phaJ gene are expressed on a plasmid, and/or, expressed by integration into the genome.

In one specific embodiment of the present invention, the introduced phaC gene and/or phaJ gene are expressed on a plasmid.

In one specific embodiment of the present invention, the introduced phaC gene and/or phaJ gene are expressed by integration into the genome.

Preferably, the target site of an sgRNA for the expression by integration into the genome comprises any one or two or more of SEQ ID NOs: 28-31.

Preferably, the expression by integration into the genome comprises integration into the genome of the halophile at G3, G4, G7, and/or G51 sites;
more preferably, the nucleotide sequence of G3 site is set forth in SEQ ID NO: 15;
more preferably, the nucleotide sequence of G4 site is set forth in SEQ ID NO: 16;
more preferably, the nucleotide sequence of G7 site is set forth in SEQ ID NO: 17;
more preferably, the nucleotide sequence of G51 site is set forth in SEQ ID NO: 18.

Preferably, the recombinant halophile comprises a single copy or two or more copies of the phaC gene and/or phaJ gene (e.g., 1, 2, 3, 4, 5, 6, 7, 8, 9, or 10 or more copies).

In one specific embodiment of the present invention, the key protein of the β-oxidation cycle pathway includes enoyl-coenzyme A hydratase. The enoyl-coenzyme A hydratase is an FadB protein; preferably, the amino acid sequence of the FadB protein comprises SEQ ID NO: 38.

Preferably, the gene coding enoyl-coenzyme A hydratase comprises an fadB gene. Preferably, the fadB gene comprises fadB1, fadB2, fadB3, fadB4, fadB5, fadB6, fadB7, fadB8, fadB9, fadB10, or fadB11.

Preferably, the nucleotide sequences of the fadB1, fadB2, fadB3, fadB4, fadB5, fadB6, fadB7, fadB8, fadB9, fadB10, or fadB11 are SEQ ID NOs: 27 or 39-48.

In one specific embodiment of the present invention, the fadB gene is fadB1, which encodes an amino acid sequence comprising SEQ ID NO: 38.

Preferably, the recombinant halophile does not express a PHA synthetase or expresses a protein with no functions or reduced functions.

Preferably, the recombinant halophile includes, but is not limited to, Halomonas bluephagenesis or a derivative thereof, Halomonas campaniensis or a derivative thereof, or Halomonas aydingkolgenesis or a derivative thereof.

In one specific embodiment of the present invention, the recombinant halophile is Halomonas bluephagenesis TD01 (CGMCC No. 4353), Halomonas campaniensis LS21 (CGMCC No. 6593), Halomonas aydingkolgenesis M1 (CGMCC No. 19880), or Halomonas bluephagenesis TDH4AB (CGMCC No. 22795).

Halomonas bluephagenesis TDH4AB is a strain capable of tolerating a low salinity obtained by the mutagenesis and screening of Halomonas bluephagenesis TD01 strain. According to the demand of specific embodiments, the recombinant halophile may also be Halomonas bluephagenesis TD01 with the endogenous PHA synthetase (SEQ ID NO: 23) knocked out, or Halomonas campaniensis LS21 with the endogenous PHA synthetase (SEQ ID NO: 24) knocked out.

Preferably, the recombinant halophile produces PHBHHx.

Preferably, the recombinant halophile can be obtained by using any preparation method in the prior art.

In a second aspect of the present invention, a method for preparing the recombinant halophile according to the first aspect is provided.

Preferably, the method comprises introducing into a halophile one or more of the following:
1) a phaC gene and/or phaJ gene;
2) an sgRNA and/or a gene encoding Cas9 protein, preferably, the sgRNA targeting an fadB gene.

Preferably, the nucleotide sequence introduced into the halophile further comprises upstream and downstream homology arms, and the upstream and downstream homology arms are derived from the fadB gene.

Preferably, the target site sequence of the sgRNA targeting the fadB gene comprises SEQ ID NO: 37.

Preferably, the phaC gene and/or phaJ gene in 1) are derived from an Aeromonas.

More preferably, the phaC gene and/or phaJ gene are each independently derived from Aeromonas caviae and Aeromonas hydriphila.

In one specific embodiment of the present invention, the phaC gene is derived from Aeromonas caviae FA440. Preferably, the nucleotide sequence comprises SEQ ID NO: 1, or has 90% or higher homology to SEQ ID NO: 1; preferably, the nucleotide sequence is set forth in SEQ ID NO: 1.

In one specific embodiment of the present invention, the phaC gene is derived from Aeromonas hydriphila 4AK4. Preferably, the nucleotide sequence comprises SEQ ID NO: 2, or has 90% or higher homology to SEQ ID NO: 2; preferably, the nucleotide sequence is set forth in SEQ ID NO: 2.

In one specific embodiment of the present invention, the phaJ gene is derived from Aeromonas caviae FA440. Preferably, the nucleotide sequence comprises SEQ ID NO: 3, or has 90% or higher homology to SEQ ID NO: 3; preferably, the nucleotide sequence is set forth in SEQ ID NO: 3.

In one specific embodiment of the present invention, the phaJ gene is derived from Aeromonas hydriphila 4AK4. Preferably, the nucleotide sequence comprises SEQ ID NO: 4, or has 90% or higher homology to SEQ ID NO: 4; preferably, the nucleotide sequence is set forth in SEQ ID NO: 4.

Preferably, the phaC gene and/or phal gene are regulated by an inducible promoter and/or a constitutive promoter.

Preferably, the target site sequence of the sgRNA targeting the fadB gene in 2) comprises SEQ ID NO: 37.

Preferably, the method comprises introducing the phaC gene and/or phaJ gene into the halophile using a vector.

Preferably, the vector comprises a promoter (e.g., an inducible promoter and/or a constitutive promoter), a ribosome-binding site (RBS), and/or a terminator (T). Preferably, the inducible promoter includes, but is not limited to, a Pₗᵤₓ promoter and/or a P_{lac} promoter.

The Pₗᵤₓ promoter is an AHL (homoserine lactone)-inducible promoter.

Preferably, the AHL induction concentration is any value in the range of 0-2 mM, preferably 0.0005-0.001 mM, e.g., 0, 0.00001, 0.00005, 0.0001, 0.0005, 0.001, 0.005, 0.01, 0.05, 0.1, 0.5, 1, 1.5, or 2 mM.

The P_{lac} promoter is an IPTG (isopropyl-β-D-thiogalactoside)-inducible promoter. Preferably, the IPTG induction concentration is any value in the range of 0-5 g/L, preferably 0.02-2 g/L, e.g., 0, 0.0005, 0.001, 0.005, 0.01, 0.02, 0.05, 0.1, 0.2, 0.5, 1, 1.5, or 2 g/L.

In one specific embodiment of the present invention, the nucleotide sequence of the Pₗᵤₓ promoter comprises SEQ ID NO: 5, or has 90% or higher homology to SEQ ID NO: 5; preferably, the nucleotide sequence is set forth in SEQ ID NO: 5.

In one specific embodiment of the present invention, the nucleotide sequence of the P_{lac} promoter comprises SEQ ID NO: 6, or has 90% or higher homology to SEQ ID NO: 6; preferably, the nucleotide sequence is set forth in SEQ ID NO: 6.

Preferably, the constitutive promoter includes, but is not limited to, a wild-type Pₚₒᵣᵢₙ or a mutant thereof.

More preferably, the Pₚₒᵣᵢₙ mutant includes, but is not limited to, one or two or more of mutant Pₚₒᵣᵢₙ₅₈, mutant Pₚₒᵣᵢₙ₄₂, mutant Pₚₒᵣᵢₙ₆₈, mutant Pₚₒᵣᵢₙ₂₇₈, mutant Pₚₒᵣᵢₙ₁₉₄, mutant Pₚₒᵣᵢₙ₂₂₁, or mutant Pₚₒᵣᵢₙ₂₀₃.

In one specific embodiment of the present invention, the nucleotide sequence of the wild-type Pₚₒᵣᵢₙ is SEQ ID NO: 7.

In one specific embodiment of the present invention, the nucleotide sequences of the mutant Pₚₒᵣᵢₙ₅₈, mutant Pₚₒᵣᵢₙ₄₂, mutant Pₚₒᵣᵢₙ₆₈, mutant Pₚₒᵣᵢₙ₂₇₈, mutant Pₚₒᵣᵢₙ₁₉₄, mutant Pₚₒᵣᵢₙ₂₂₁, or mutant Pₚₒᵣᵢₙ₂₀₃ comprise SEQ ID NOs: 8-14, or have 90% or higher homology to SEQ ID NOs: 8-14; preferably, the nucleotide sequences are set forth in SEQ ID NOs: 8-14.

Preferably, the sequence of the ribosome-binding site comprises SEQ ID NO: 19 (ribosome-binding site 1) or SEQ ID NO: 20 (ribosome-binding site 2), or has 90% or higher homology to SEQ ID NO: 19 or 20, preferably, the sequence of the ribosome-binding site is set forth in SEQ ID NO: 19 or 20.

Preferably, the sequence of the terminator comprises SEQ ID NO: 21 (terminator 1) or SEQ ID NO: 22 (terminator 2), or has 90% or higher homology to SEQ ID NO: 21 or 22, preferably, the sequence of the terminator is set forth in SEQ ID NO: 21 or 22.

Preferably, the introduced phaC gene and/or phaJ gene are expressed on a plasmid, and/or, expressed by integration into the genome.

In one specific embodiment of the present invention, the introduced phaC gene and/or phaJ gene are expressed on a plasmid.

In one specific embodiment of the present invention, the introduced phaC gene and/or phaJ gene are expressed by integration into the genome.

Preferably, the promoter is an inducible promoter. Preferably, the vector may or may not comprise a terminator.

In one specific embodiment of the present invention, the order of the phaC gene, the phaJ gene, the promoter, the ribosome-binding site, and the terminator in the vector is: the inducible promoter, the ribosome-binding site 1, the phaC gene, the terminator 1, the inducible promoter, the ribosome-binding site 2, the phaJ gene, and the terminator 2.

In one specific embodiment of the present invention, the vector does not comprise a terminator, and the order of the phaC gene, the phaJ gene, the promoter, and the ribosome-binding site in the vector is: the inducible promoter, the ribosome-binding site 1, the phaC gene, the inducible promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the vector are interchangeable, as long as the normal expression is possible.

Preferably, the inducible promoter includes, but is not limited to, a Pₗᵤₓ promoter and/or a P_{lac} promoter.

Preferably, the promoter is a constitutive promoter. Preferably, the vector may or may not comprise a terminator.

In one specific embodiment of the present invention, the order of the phaC gene, the phaJ gene, the promoter, the ribosome-binding site, and the terminator in the vector is: the constitutive promoter, the ribosome-binding site 1, the phaC gene, the terminator 1, the constitutive promoter, the ribosome-binding site 2, the phal gene, and the terminator 2.

In one specific embodiment of the present invention, the vector does not comprise a terminator, and the order of the phaC gene, the phal gene, the promoter, and the ribosome-binding site in the vector is: the constitutive promoter, the ribosome-binding site 1, the phaC gene, the constitutive promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the vector are interchangeable, as long as the normal expression is possible.

Preferably, the constitutive promoter includes, but is not limited to, a wild-type Pₚₒᵣᵢₙ and/or a mutant thereof.

Preferably, the vector comprises SEQ ID NO: 32, or, comprises a nucleotide sequence having at least 90% identity to the nucleotide sequence set forth in SEQ ID NO: 32. Preferably, the introduction comprises introducing the phaC gene and/or phaJ gene into any one, two, three, or four of G3 (SEQ ID NO: 15), G4 (SEQ ID NO: 16), G7 (SEQ ID NO: 17), or G51 (SEQ ID NO: 18) site in the genome of the halophile.

Preferably, the introduced phaC gene and/or phaJ gene include a single copy or two or more copies (e.g., 1, 2, 3, 4, 5, 6, 7, 8, 9, or 10 or more copies).

According to the demand of specific embodiments, the single copy or the two or more copies can be achieved by introducing the target gene (the phaC gene and/or phaJ gene in the present application) into one or more sites in the genome. For example, the target gene can be introduced into the genome at any one of G3 (SEQ ID NO: 15), G4 (SEQ ID NO: 16), G7 (SEQ ID NO: 17), and/or G51 (SEQ ID NO: 18) sites to achieve a single copy; the target gene can be introduced at any two of G3 (SEQ ID NO: 15), G4 (SEQ ID NO: 16), G7 (SEQ ID NO: 17), and/or G51 (SEQ ID NO: 18) sites in the genome to achieve two copies; the target gene can be introduced into any three of G3 (SEQ ID NO: 15), G4 (SEQ ID NO: 16), G7 (SEQ ID NO: 17), and/or G51 (SEQ ID NO: 18) sites in the genome to achieve three copies; the target gene can be introduced into the genome at G3 (SEQ ID NO: 15), G4 (SEQ ID NO: 16), G7 (SEQ ID NO: 17), and G51 (SEQ ID NO: 18) to achieve four copies.

Preferably, the two or more copies can be two or more copies (e.g., 2, 3, 4, 5, 6, 7, 8, 9, or 10 or more copies) achieved by multiple integrations at a single site in the genome, or, three or more copies (e.g., 3, 4, 5, 6, 7, 8, 9, or 10 or more copies) achieved by the combination of multiple integrations at a single site and separate integrations at multiple sites in the genome.

In one specific embodiment of the present invention, the introduction comprises introducing the phaC gene and/or phaJ gene into any one of G3, G4, G7, or G51 site in the genome of the halophile.

In one specific embodiment of the present invention, the introduction comprises introducing the phaC gene and/or phaJ gene into any one of G3 and G4 sites, G3 and G7 sites, G3 and G51 sites, G4 and G7 sites, G4 and G51 sites, or G7 and G51 sites in the genome of the halophile.

In one specific embodiment of the present invention, the introduction comprises introducing the phaC gene and/or phaJ gene into any one of G3, G4 and G7 sites, G3, G4 and G51 sites, G3, G7 and G51 sites, or G4, G7 and G51 sites in the genome of the halophile.

In one specific embodiment of the present invention, the introduction comprises introducing the phaC gene and/or phaJ gene into G3, G4, G7, and G51 sites in the genome of the halophile.

Preferably, the vector comprises the nucleotide sequence set forth in any one of SEQ ID NOs: 33-36, or, comprises a nucleotide sequence having at least 90% identity to the nucleotide sequence set forth in any one of SEQ ID NOs: 33-36.

In one specific embodiment of the present invention, the method comprises:
1) constructing phaC and phal gene expression plasmids regulated by an inducible and/or constitutive promoter, wherein preferably, the inducible promoter includes, but is not limited to, a Pₗᵤₓ promoter (SEQ ID NO: 5) and/or a P_{lac} promoter (SEQ ID NO: 6);
preferably, the constitutive promoter includes, but is not limited to, wild-type Pₚₒᵣᵢₙ (SEQ ID NO: 7) or a mutant thereof; more preferably, the Pₚₒᵣᵢₙ mutant includes, but is not limited to, mutant Pₚₒᵣᵢₙ₅₈ (SEQ ID NO: 8), mutant Pₚₒᵣᵢₙ₄₂ (SEQ ID NO: 9), mutant Pₚₒᵣᵢₙ₆₈ (SEQ ID NO: 10), mutant Pₚₒᵣᵢₙ₂₇₈ (SEQ ID NO: 11), mutant Pₚₒᵣᵢₙ₁₉₄ (SEQ ID NO: 12), mutant Pₚₒᵣᵢₙ₂₂₁ (SEQ ID NO: 13), and mutant Pₚₒᵣᵢₙ₂₀₃ (SEQ ID NO: 14); the phaC and phaJ genes are derived from an Aeromonas;
2) transforming a halophile with the phaC and phaJ gene expression plasmids obtained in 1) by conjugation; preferably, the halophile includes, but is not limited to, Halomonas bluephagenesis or a derivative thereof, Halomonas campaniensis or a derivative thereof, and Halomonas aydingkolgenesis or a derivative thereof, more preferably Halomonas bluephagenesis TD01 (CGMCC No. 4353), Halomonas campaniensis LS21 (CGMCC No. 6593), Halomonas aydingkolgenesis M1 (CGMCC No. 19880), and Halomonas bluephagenesis TDH4AB (CGMCC No. 22795).

Halomonas bluephagenesis TDH4AB is a strain capable of tolerating a low salinity obtained by the mutagenesis and screening of Halomonas bluephagenesis TD01 strain. According to the demand of specific embodiments, the recombinant microbe may also be Halomonas bluephagenesis TD01 with the endogenous PHA synthetase (SEQ ID NO: 23) knocked out, or Halomonas campaniensis LS21 with the endogenous PHA synthetase (SEQ ID NO: 24) knocked out.

In one specific embodiment of the present invention, the method comprises:
1) knocking out the fadB gene in a halophile, preferably, knocking out the fadB gene by using a CRISPR/Cas9 genome editing method, wherein preferably, the CRISPR/Cas9 genome editing method comprises using an sgRNA, and preferably the nucleotide sequence of the target site sequence of the sgRNA comprises SEQ ID NO: 37.
2) constructing expression plasmids of the phaC and phal genes regulated by an inducible and/or constitutive promoter, wherein preferably, the inducible promoter includes, but is not limited to, a Pₗᵤₓ promoter (SEQ ID NO: 5) and/or a P_{lac} promoter (SEQ ID NO: 6);
preferably, the constitutive promoter includes, but is not limited to, wild-type Pₚₒᵣᵢₙ (SEQ ID NO: 7) or a mutant thereof; more preferably, the Pₚₒᵣᵢₙ mutant includes, but is not limited to, mutant Pₚₒᵣᵢₙ₅₈ (SEQ ID NO: 8), mutant Pₚₒᵣᵢₙ₄₂ (SEQ ID NO: 9), mutant Pₚₒᵣᵢₙ₆₈ (SEQ ID NO: 10), mutant Pₚₒᵣᵢₙ₂₇₈ (SEQ ID NO: 11), mutant Pₚₒᵣᵢₙ₁₉₄ (SEQ ID NO: 12), mutant Pₚₒᵣᵢₙ₂₂₁ (SEQ ID NO: 13), and mutant Pₚₒᵣᵢₙ₂₀₃ (SEQ ID NO: 14). The phaC and phaJ genes are derived from an Aeromonas.
3) transforming the halophile with fadB gene knockout obtained in 1) with the phaC and phaJ gene expression plasmids obtained in 2) by conjugation; preferably, the halophile includes, but is not limited to, Halomonas bluephagenesis or a derivative thereof, Halomonas campaniensis or a derivative thereof, and Halomonas aydingkolgenesis or a derivative thereof, more preferably Halomonas bluephagenesis TD01 (CGMCC No. 4353), Halomonas campaniensis LS21 (CGMCC No. 6593), Halomonas aydingkolgenesis M1 (CGMCC No. 19880), and Halomonas bluephagenesis TDH4AB (CGMCC No. 22795); Halomonas bluephagenesis TDH4AB is a strain capable of tolerating a low salinity obtained by the mutagenesis and screening of Halomonas bluephagenesis TD01 strain.

According to the demand of specific embodiments, the recombinant microbe may also be Halomonas bluephagenesis TD01 with the endogenous PHA synthetase (SEQ ID NO: 23) knocked out, or Halomonas campaniensis LS21 with the endogenous PHA synthetase (SEQ ID NO: 24) knocked out.

In one specific embodiment of the present invention, the method comprises:
1) constructing phaC and phal gene expression plasmids regulated by an inducible and/or constitutive promoter, wherein preferably, the inducible promoter includes, but is not limited to, a Pₗᵤₓ promoter (SEQ ID NO: 5) and/or a P_{lac} promoter (SEQ ID NO: 6);
preferably, the constitutive promoter includes, but is not limited to, wild-type Pₚₒᵣᵢₙ (SEQ ID NO: 7) or a mutant thereof; more preferably, the Pₚₒᵣᵢₙ mutant includes, but is not limited to, mutant Pₚₒᵣᵢₙ₅₈ (SEQ ID NO: 8), mutant Pₚₒᵣᵢₙ₄₂ (SEQ ID NO: 9), mutant Pₚₒᵣᵢₙ₆₈ (SEQ ID NO: 10), mutant Pₚₒᵣᵢₙ₂₇₈ (SEQ ID NO: 11), mutant Pₚₒᵣᵢₙ₁₉₄ (SEQ ID NO: 12), mutant Pₚₒᵣᵢₙ₂₂₁ (SEQ ID NO: 13), and mutant Pₚₒᵣᵢₙ₂₀₃ (SEQ ID NO: 14); the phaC and phaJ genes are derived from an Aeromonas;
2) transforming a halophile with the phaC and phaJ gene expression plasmids obtained in 1) by conjugation; preferably, the halophile includes, but is not limited to, Halomonas bluephagenesis or a derivative thereof, Halomonas campaniensis or a derivative thereof, and Halomonas aydingkolgenesis or a derivative thereof, more preferably Halomonas bluephagenesis TD01 (CGMCC No. 4353), Halomonas campaniensis LS21 (CGMCC No. 6593), Halomonas aydingkolgenesis M1 (CGMCC No. 19880), and Halomonas bluephagenesis TDH4AB (CGMCC No. 22795); Halomonas bluephagenesis TDH4AB is a strain capable of tolerating a low salinity obtained by the mutagenesis and screening of Halomonas bluephagenesis TD01 strain.
3) knocking out the fadB gene in the halophile transformed with the phaC and phaJ gene expression plasmids by conjugation obtained in 2), preferably, knocking out the fadB gene by using a CRISPR/Cas9 genome editing method, wherein preferably, the CRISPR/Cas9 genome editing method comprises using an sgRNA, and preferably the nucleotide sequence of the target site sequence of the sgRNA comprises SEQ ID NO: 37.

Preferably, the transformation by conjugation is an integration into the genome of the halophile, more preferably at one, two, three, or four of G3, G4, G7, or G51 site of the genome of the halophile;

preferably, the phaC and phaJ genes conjugated in the genome of the halophile are in a single copy or in two or more copies.

In a third aspect of the present invention, a recombinant halophile obtained by the method according to the second aspect is provided.

In a fourth aspect of the present invention, a vector is provided.

Preferably, the vector comprises:
1) a phaC gene and/or phaJ gene; and/or
2) an sgRNA and/or a gene encoding Cas9 protein, preferably, the sgRNA targeting an fadB gene,
wherein preferably, the vector further comprises upstream and downstream homology arms, and the upstream and downstream homology arms are derived from the fadB gene.

Preferably, the target site sequence of the sgRNA targeting the fadB gene comprises SEQ ID NO: 37.

Preferably, the phaC gene and/or phaJ gene in 1) are derived from an Aeromonas. Preferably, the phaC gene and/or phaJ gene are regulated by an inducible promoter and/or a constitutive promoter.

Preferably, the inducible promoter includes, but is not limited to, Pₗᵤₓ and/or P_{lac}; preferably, the constitutive promoter includes, but is not limited to, Pₚₒᵣᵢₙ or a mutant thereof;
more preferably, the Pₚₒᵣᵢₙ mutant includes one or two or more of mutant Pₚₒᵣᵢₙ₅₈, mutant Pₚₒᵣᵢₙ₄₂, mutant Pₚₒᵣᵢₙ₆₈, mutant Pₚₒᵣᵢₙ₂₇₈, mutant Pₚₒᵣᵢₙ₁₉₄, mutant Pₚₒᵣᵢₙ₂₂₁, or mutant Pₚₒᵣᵢₙ₂₀₃.

Preferably, the vector further comprises a ribosome-binding site and/or a terminator, and more preferably, the ribosome-binding site and/or the terminator may be any ribosome-binding site and/or terminator sequence in the prior art.

Preferably, the promoter is an inducible promoter.

Preferably, the vector may or may not comprise a terminator.

In one specific embodiment of the present invention, the order of the phaC gene, the phaJ gene, the promoter, the ribosome-binding site, and the terminator in the vector is: the inducible promoter, the ribosome-binding site 1, the phaC gene, the terminator 1, the inducible promoter, the ribosome-binding site 2, the phaJ gene, and the terminator 2.

In one specific embodiment of the present invention, the vector does not comprise a terminator, and the order of the phaC gene, the phaJ gene, the promoter, and the ribosome-binding site in the vector is: the inducible promoter, the ribosome-binding site 1, the phaC gene, the inducible promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the vector are interchangeable, as long as the normal expression is possible.

Preferably, the promoter is a constitutive promoter.

Preferably, the vector may or may not comprise a terminator.

In one specific embodiment of the present invention, the order of the phaC gene, the phaJ gene, the promoter, the ribosome-binding site, and the terminator in the vector is: the constitutive promoter, the ribosome-binding site 1, the phaC gene, the terminator 1, the constitutive promoter, the ribosome-binding site 2, the phal gene, and the terminator 2.

In one specific embodiment of the present invention, the vector does not comprise a terminator, and the order of the phaC gene, the phal gene, the promoter, and the ribosome-binding site in the vector is: the constitutive promoter, the ribosome-binding site 1, the phaC gene, the constitutive promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the vector are interchangeable, as long as the normal expression is possible.

Preferably, the vector comprises SEQ ID NO: 32, or, comprises a nucleotide sequence having at least 90% identity to the nucleotide sequence set forth in SEQ ID NO: 32. Preferably, the vector comprises any nucleotide sequence of SEQ ID NOs: 33-36, or, comprises a nucleotide sequence having at least 90% identity to the nucleotide sequence set forth in any one of SEQ ID NOs: 33-36.

Preferably, the sgRNA described in 2), upstream and downstream homology arms, and/or the gene coding Cas9 protein can be located at any position in the vector as long as the normal homologous recombination is possible.

In a fifth aspect of the present invention, an expression system comprising the vector according to the fourth aspect is provided.

Preferably, the ribosome-binding site in the expression system includes ribosome-binding site 1 (SEQ ID NO: 19).

Preferably, the ribosome-binding site in the expression system includes ribosome-binding site 2 (SEQ ID NO: 20).

Preferably, the terminator in the expression system includes terminator 1 (SEQ ID NO: 21).

Preferably, the terminator in the expression system includes terminator 2 (SEQ ID NO: 22).

Preferably, the expression system may be an inducible expression system or a constitutive expression system.

Preferably, the expression system is an inducible expression system, and more preferably, the inducible expression system uses an inducible promoter.

Preferably, the inducible expression system may or may not comprise a terminator.

In one specific embodiment of the present invention, the inducible expression system comprises the following elements arranged in the following order: the inducible promoter, the ribosome-binding site 1, the phaC gene, the terminator 1, the inducible promoter, the ribosome-binding site 2, the phaJ gene, and the terminator 2. In one specific embodiment of the present invention, the inducible expression system does not comprise a terminator, but comprises the following elements arranged in the following order: the inducible promoter, the ribosome-binding site 1, the phaC gene, the inducible promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the inducible expression system are interchangeable, as long as the normal expression is possible.

Preferably, the inducible promoter includes, but is not limited to, Pₗᵤₓ and/or P_{lac}. Preferably, the expression system is a constitutive expression system, and more preferably, the constitutive expression system uses a constitutive promoter. Preferably, the constitutive expression system may or may not comprise a terminator. In one specific embodiment of the present invention, the constitutive expression system comprises the following elements arranged in the following order: the constitutive promoter, the ribosome-binding site 1, the phaC gene, the terminator 1, the constitutive promoter, the ribosome-binding site 2, the phaJ gene, and the terminator 2.

In one specific embodiment of the present invention, the constitutive expression system does not comprise a terminator, but comprises the following elements arranged in the following order: the constitutive promoter, the ribosome-binding site 1, the phaC gene, the constitutive promoter, the ribosome-binding site 2, and the phaJ gene.

According to the demand of specific embodiments, the orders of the phaC gene and phaJ gene, the ribosome-binding site 1 and the ribosome-binding site 2, and the terminator 1 and the terminator 2 in the constitutive expression system are interchangeable.

Preferably, the constitutive promoter includes, but is not limited to, a wild-type Pₚₒᵣᵢₙ and/or a mutant thereof.

Preferably, the constitutive expression system comprises SEQ ID NO: 32, or, comprises a nucleotide sequence having at least 90% identity to the nucleotide sequence set forth in SEQ ID NO: 32.

Preferably, the constitutive expression system comprises any nucleotide sequence of SEQ ID NOs: 33-36, or, comprises a nucleotide sequence having at least 90% identity to the nucleotide sequence set forth in any one of SEQ ID NOs: 33-36.

In a sixth aspect of the present invention, a cell comprising the vector described above and/or the expression system described above is provided.

In a seventh aspect of the present invention, use of the vector described above, the expression system described above, and/or the cell described above in producing a PHA (particularly PHBHHx) is provided.

In an eighth aspect of the present invention, a fermentation method is provided, comprising fermentatively culturing the recombinant halophile described above, and/or, a recombinant halophile obtained by the method described above.

Preferably, the fermentation method does not require sterilization.

Preferably, the fermentation product includes, but is not limited to, PHBHHx.

Preferably, the fermentation medium is a conventional medium or the composition of the medium may be properly adjusted to accommodate the survival and production of the microbe.

Preferably, the fermentation conditions may be properly adjusted depending on the specific recombinant microbe.

Preferably, the fermentation apparatus may be a shake flask, a small fermenter, a pilot fermenter, or a large fermenter for industrial mass production.

Preferably, the carbon source in the fermentation process includes, but is not limited to, one or two or more of hexanoic acid, a hexanoate, or glucose.

Preferably, the carbon source in the fermentation process includes one or two of: 1) hexanoic acid and/or a hexanoate, or, 2) glucose.

More preferably, the hexanoate includes, but is not limited to, sodium hexanoate and/or potassium hexanoate.

In one specific embodiment of the present invention, the carbon source in the fermentation process is hexanoic acid.

In one specific embodiment of the present invention, the carbon source in the fermentation process is hexanoic acid and a hexanoate (preferably sodium hexanoate). In one specific embodiment of the present invention, the carbon source in the fermentation process is a hexanoate (preferably sodium hexanoate).

In one specific embodiment of the present invention, the carbon source in the fermentation process is hexanoic acid and glucose.

In one specific embodiment of the present invention, the carbon source in the fermentation process is a hexanoate (preferably sodium hexanoate) and glucose.

In one specific embodiment of the present invention, the carbon source in the fermentation process is hexanoic acid, a hexanoate (preferably sodium hexanoate), and glucose.

In a ninth aspect of the present invention, a method for preparing a recombinant microbe is provided, comprising introducing phaC and phaJ gene expression plasmids into a halophile.

Preferably, the method further comprises knocking out an fadB gene in the halophile. Preferably, the phaC and phal gene expression plasmids, the fadB gene knockout, and the halophile are as defined in the second aspect of the present invention.

In a tenth aspect of the present invention, a method for preparing a recombinant microbe is provided, comprising knocking out an fadB gene in a halophile.

Preferably, the method further comprises introducing phaC and phal gene expression plasmids into the halophile.

Preferably, the phaC and phaJ gene expression plasmids, the fadB gene knockout, and the halophile are as defined in the second aspect of the present invention.

In an eleventh aspect of the present invention, a method for producing PHBHHx is provided.

Preferably, the method comprises fermentatively culturing the recombinant halophile described above, and/or, a recombinant halophile obtained by the method described above.

Preferably, the carbon source in the fermentation process includes, but is not limited to, one or two or more of hexanoic acid, a hexanoate, or glucose.

Preferably, the carbon source in the fermentation process includes one or two of: 1) hexanoic acid and/or a hexanoate, or, 2) glucose.

More preferably, the hexanoate includes, but is not limited to, sodium hexanoate and/or potassium hexanoate.

In one specific embodiment of the present invention, the carbon source in the fermentation process is hexanoic acid.

In one specific embodiment of the present invention, the carbon source in the fermentation process is hexanoic acid and a hexanoate (preferably sodium hexanoate). In one specific embodiment of the present invention, the carbon source in the fermentation process is a hexanoate (preferably sodium hexanoate).

In one specific embodiment of the present invention, the carbon source in the fermentation process is hexanoic acid and glucose.

In one specific embodiment of the present invention, the carbon source in the fermentation process is a hexanoate (preferably sodium hexanoate) and glucose.

In one specific embodiment of the present invention, the carbon source in the fermentation process is hexanoic acid, a hexanoate (preferably sodium hexanoate), and glucose.

In a twelfth aspect of the present invention, a method for increasing the molar proportion of 3HHx monomer in PHBHHx produced by a halophile is provided. Preferably, the method comprises:
1) fermentatively culturing the recombinant halophile described above, and/or, a recombinant halophile obtained by the method described above; and/or, 2) adjusting the carbon source in the fermentation process.

Preferably, the carbon source in 2) includes, but is not limited to, one or two or more of hexanoic acid, hexanoate, or glucose.

More preferably, the hexanoate includes, but is not limited to, sodium hexanoate and/or potassium hexanoate.

In one specific embodiment of the present invention, the carbon source in the fermentation process is hexanoic acid.

In one specific embodiment of the present invention, the carbon source in the fermentation process is hexanoic acid and sodium hexanoate. In one specific embodiment of the present invention, the carbon source in the fermentation process is sodium hexanoate.

In one specific embodiment of the present invention, the carbon source in the fermentation process is hexanoic acid and glucose.

In one specific embodiment of the present invention, the carbon source in the fermentation process is sodium hexanoate and glucose.

In one specific embodiment of the present invention, the carbon source in the fermentation process is hexanoic acid, sodium hexanoate, and glucose.

Preferably, the adjustment of the carbon source in the fermentation process comprises adjusting the variety of the carbon source added in the fermentation process.

Preferably, the adjustment of the carbon source in the fermentation process comprises adjusting the ratio of glucose to hexanoic acid or the hexanoate in the carbon source added in the fermentation process.

Preferably, the ratio of glucose to hexanoic acid or the hexanoate in the carbon source added in the fermentation process is any value in the range of (0-50) g/L:(0-20) g/L, preferably (0.1-30) g/L:(0.1-7.5) g/L, e.g., 0:7.5, 0.1:0.1, 0.1:7.5, 30:0.1, 0:20, 10:7.5, 20:7.5, 30:7.5, 30:0, 50:0, or 50:20.

Preferably, according to the method, the adjustable range of the molar proportion of 3HHx in PHBHHx is: any value of 0-80 mol %, preferably any value of 3-50 mol %. For example, the molar proportion of 3HHx in PHBHHx may be adjusted to 0, 3, 3.94, 4.38, 5, 5.81, 6.58, 6.98, 7.18, 7.82, 7.88, 8, 8.07, 8.17, 8.21, 9, 9.78, 10, 10.01, 10.12, 10.31, 10.76, 11, 11.22, 11.96, 11.97, 12, 12.77, 12.86, 13, 13.67, 13.82, 13.94, 14, 14.58, 14.67, 15, 18, 18.25, 18.27, 18.39, 20, 20.11, 20.19, 22.09, 23.05, 23.31, 23.95, 24.05, 24.35, 24.43, 25, 25.08, 26.05, 26.66, 26.89, 27.08, 27.71, 27.97, 28.15, 29.89, 30, 35, 38, 38.17, 39, 40, 45, 49.05, or 50 mol %.

The term “comprises”, “comprising”, “include”, “including”, “contain” or “containing” of the present invention is an open-ended description that includes the stated specific components or steps and does not essentially influence other specific components or steps; when the term is used to describe a sequence of a protein or nucleic acid, the protein or nucleic acid may consist of the sequence, or may have additional amino acids or nucleotides at one or two ends of the protein or nucleic acid, while retaining the activity described herein.

The term “and/or” described herein encompasses all combinations of the items connected by the term, and each combination should be deemed to have been individually listed herein. For example, “A and/or B” encompasses “A”, “A and B”, and “B”. For another example, “A, B, and/or C” encompasses “A”, “B”, “C”, “A and B”, “A and C”, “B and C”, and “A and B and C”.

Abbreviations in the present application are shown in Table 1:

TABLE 1
Abbreviations in the present application

| Abbreviation | Full version |
|---|---|
| fadL | membrane transport protein-coding gene |
| fadD | Acyl-coenzyme A synthetase-coding gene |
| fadE | Acyl-coenzyme A dehydrogenase-coding gene |
| phaJ | Enoyl-coenzyme A hydratase-coding gene |
| fadB | Enoyl-coenzyme A hydratase-coding gene |
| fadA | Ketoacyl-coenzyme A thiolase-coding gene |
| phaA | β-Ketothiolase-coding gene |
| phaB | NADPH/NADH-dependent acetoacetyl reductase-coding |
| | gene |
| phaC | PHA hydratase-coding gene |
| RBS | Ribosome-binding site |
| Terminator | Terminator |
| PHBHHx | 3-hydroxybutyric acid/3-hydroxyhexanoic acid copolymer |
| P3HB | Poly-3-hydroxybutyrate |

The foregoing is merely a summary of some aspects of the present invention, and is not intended to limit and should not be construed as limiting the present invention in any way.

All patents and publications mentioned in this specification are herein incorporated by reference in their entirety. Those skilled in the art will appreciate that certain changes may be made to the present invention without departing from the conception or scope of the present invention. The following examples are intended to further illustrate the present invention in detail and should not be construed as limiting the scope of the present invention or the specific methods described herein.

### BRIEF DESCRIPTION OF THE DRAWINGS

FIG. 1 illustrates a schematic of the PHBHHx synthetic pathway, wherein fad is an membrane transport protein-coding gene; fadD is an acyl-coenzyme A synthetase-coding gene; fadE is an acyl-coenzyme A dehydrogenase-coding gene; phaJ is an enoyl-coenzyme A hydratase-coding gene; fadB is an enoyl-coenzyme A hydratase-coding gene; fadA is a ketoacyl-coenzyme A thiolase-coding gene; phaA is a β-ketothiolase-coding gene; phaB is an NADPH/NADH-dependent acetoacetyl reductase-coding gene; phaC is a PHA hydratase-coding gene;

FIG. 2 illustrates the fluorescence intensity at different AHL concentrations in halophile TDC-pDI-dfp;

FIG. 3 illustrates the fluorescence intensity at different IPTG concentrations in halophile TDC-pDI-dfp;

FIG. 4 illustrates a schematic of the PHBHHx production at different inducer concentration combinations in halophile TDC-pDI-CJ_{FA440};

FIG. 5 illustrates a schematic of the PHBHHx production in a recombinant halophile with the functional module integrated at a single site;

FIG. 6 illustrates a schematic of the PHBHHx production in a recombinant halophile with the functional module integrated at multiple sites;

FIG. 7 illustrates a schematic of the PHBHHx fermentative production in recombinant halophile TDC-G34 with sodium hexanoate as the carbon source;

FIG. 8 illustrates a schematic of the PHA production in recombinant halophile TDC-G34 with glucose as the carbon source;

FIG. 9 illustrates a schematic of the PHBHHx production in recombinant halophile TDC-G34 with a mixed carbon source.

### DETAILED DESCRIPTION

The embodiments in the examples of the present invention will be described clearly and completely below with reference to the drawings. It is apparent that the described examples are only a part of the examples of the present invention, but not all of them. Based on the embodiments of the present invention, all other examples obtained by those of ordinary skills in the art without creative work shall fall within the protection scope of the present invention.

Hereinafter, the present invention will be described in detail by means of examples. The experimental methods used in the following examples are all conventional methods unless otherwise specified.

Reagents, materials, and the like used in the following examples are commercially available unless otherwise specified.

Escherichia coli was cultured in LB medium containing: 10 g/L of sodium chloride, 10 g/L of peptone, and 5 g/L of a yeast extract.

The halophile was cultured in LB60 medium unless otherwise specified. The 60 LB composition was substantially identical to the LB medium except that the concentration of sodium chloride was adjusted to 60 g/L.

The culture conditions of E. coli and the halophile were 37° C. and 200 rpm.

The halophile gene editing technique used in the present invention is CRISPR/Cas9 gene editing, including endogenous DNA knockout and heterologous DNA integration, see Qin et. al., CRISPR/Cas9 editing genome of extremophile Halomonas spp. Metabolic Engineering. 47 (2018) 219-229.

Medium for shake flask fermentative production of PHBHHx:

60 LB fermentation medium: 60 g/L of sodium chloride, 5 g/L of a yeast extract, 10 g/L of tryptone, and 0.1-50 g/L of related carbon source combination.

Basic culture medium: 0.1-50 g/L of related carbon source combination, 55-70 g/L of sodium chloride, 1-10 g/L of yeast extract, 3-6 g/L of urea, 1.5-5.2 g/L of potassium dihydrogen phosphate, 0.2-0.4 g/L of magnesium sulfate, 8.5-10 g/L of disodium hydrogen phosphate, 7-15 mL/L of component III, and 1-5 mL/L of component IV. Component III: 5 g/L of ferric ammonium citrate, 2 g/L of calcium chloride dihydrate, and 41.7 mL of concentrated hydrochloric acid (12 mol/L), diluted to 1000 mL with water.

Component IV: 100 mg/L of zinc sulfate heptahydrate, 30 mg/L of manganese chloride tetrahydrate, 300 mg/L of boric acid, 200 mg/L of cobalt chloride hexahydrate, 10 mg/L of anhydrous copper sulfate, 20 mg/L of nickel chloride hexahydrate, and 30 mg/L of sodium molybdate dihydrate.

The above culture media can be prepared by standard preparation methods.

### Cell Dry Weight Measurement:

The weight of a 50-mL empty centrifuge tube was measured; a certain volume of cells cultured from a shake flask or a fermenter was added to the centrifuge tube and centrifuged for 10 min at 10000×g, the supernatant was discarded, and the cells were collected; the cells were resuspended in an appropriate amount of deionized water and centrifuged for 10 min at 10000×g, the supernatant was discarded, and the cells were collected again; the obtained cell precipitate was frozen in a cryogenic freezer at −80° C. for more than 3 h, and processed in a vacuum lyophilizer until the weight no longer changed; the total weight of the centrifuge tube and the dried cells in the tube was measured; the dry cell weight was determined by the difference method.

### PHBHHx Assay:

30-40 mg of dried cells or about 15 mg of standard sample (P3HB or methyl 3-hydroxyhexanoate) was added in an esterification tube, 2 mL of esterification solution (3% (v/v) of concentrated sulfuric acid and 0.5 g/L of benzoic acid in chromatographically pure methanol solution) and 2 mL of chloroform were added, and the tube was capped and sealed; the system was reacted thermostatically at 100° C. for 4 h and then cooled to room temperature; 1 mL of deionized water was added into each tube, and the tube was shaken to mix uniformly, and let stand until the liquid was completely layered; a proper amount of the lower chloroform phase sample was taken by pipetting for GC analysis; the GC analysis procedures were: the column temperature was increased from room temperature to 80° C. and then held for 90 s, increased to 140° C. at a rate of 0.5° C./s and held for 0 s, increased to 240° C. at a rate of 0.7° C./s and held for 120 s for analysis, and then reduced to room temperature to finish the analysis; the PHA was quantitatively analyzed according to the peak area value via internal standard normalization, and the molar proportions of PHA, 3HB, and 3HHx to the dry cell weight were calculated.

The embodiments described herein are intended to describe in detail the construction methods and use of the recombinant halophile for producing PHBHHx, and are thus illustrative rather than limiting. A number of other examples can be given according to the defined scope. Therefore, variations and modifications without departing from the general concept of the present invention shall fall within the scope of the present invention.

### Example 1: Construction of Recombinant Halophile Containing Inducible phaC-phaJ Expression Plasmid Module for PHBHHx Production

A dual-inducible phaC-phaJ heterologous expression plasmid module was constructed and transformed into the halophile by conjugation to give a recombinant halophile containing the functional module. Fermentative PHBHHx production was conducted in shake flasks by adding different concentrations of related carbon sources and different concentrations of inducers.

The specific implementation procedures are as follows:

(1) Construction of Inducible phaC-phaJ Expression Plasmid

On the basis of Aeromonas caviae FA440 and/or Aeromonas hydriphila 4AK4 genomes as the templates, specific primers were designed for PCR amplification of the phaC gene elements and the phaJ gene elements. The AHL-inducible promoter elements (containing AHL promoter and regulatory module) and the IPTG-inducible promoter elements (containing IPTG promoter and regulatory module) were synthesized directly. The ribosome-binding sites RBS1 and RBS2 were synthesized directly. Terminator 1 and terminator 2 were synthesized directly. The expression module “Pₗᵤₓ-RBS1-phaC-T1-P_{lac}-RBS2-phaJ-T2” was integrated into the multiple enzyme cleavage sites of the low-copy plasmid pSEVA321 using Gibson Assembly technology. After colony PCR and gene sequencing confirmation, the successfully constructed plasmid was designated as: pDI-CJ.

Specifically, when phaC and phaJ in plasmid pDI-CJ were both derived from Aeromonas caviae FA440, the plasmid was designated as: pDI-CJ_{FA440};

Specifically, when phaC and phaJ in plasmid pDI-CJ were both derived from Aeromonas hydriphila 4AK4, the plasmid was designated as: pDI-CJ_{4AK4};

(2) Construction of Recombinant Halophile Containing Plasmid pDI-CJ

Firstly, E. coli S17-1 was transformed with the constructed plasmid pDI-CJ, and then different halophiles were transformed with the plasmid pDI-CJ by conjugation, so as to give different recombinant halophile strains.

Specifically, the recombinant halophile obtained by transforming halophile H. bluephagenesis TD01 with the plasmid pDI-CJ_{4AK4} was designated as TD-pDI-CJ_{4AK4}; Specifically, the recombinant halophile obtained by transforming PHA synthetase-deficient halophile H. bluephagenesis TD01 with the plasmid pDI-CJ_{4AK4} was designated as TDC-pDI-CJ_{4AK4};
Specifically, the recombinant halophile obtained by transforming halophile H. campaniensis LS21 with the plasmid pDI-CJ_{4AK4} was designated as LS-pDI-CJ_{4AK4}; Specifically, the recombinant halophile obtained by transforming PHA synthetase-deficient halophile H. campaniensis LS21 with the plasmid pDI-CJ_{4AK4} was designated as LSC-pDI-CJ_{4AK4};
Specifically, the recombinant halophile obtained by transforming halophile Halomonas aydingkolgenesis M1 with the plasmid pDI-CJ_{4AK4} was designated as M1-pDI-CJ_{4AK4}; Specifically, the recombinant halophile obtained by transforming halophile H. bluephagenesis TD01 with the plasmid pDI-CJ_{FA440} was designated as TD-pDI-CJ_{FA440}. Specifically, the recombinant halophile obtained by transforming PHA synthetase-deficient halophile H. bluephagenesis TD01 with the plasmid pDI-CJ_{FA440} was designated as TDC-pDI-CJ_{FA440};
Specifically, the recombinant halophile obtained by transforming halophile H. campaniensis LS21 with the plasmid pDI-CJ_{FA440} was designated as LS-pDI-CJ_{FA440}; Specifically, the recombinant halophile obtained by transforming PHA synthetase-deficient halophile H. campaniensis LS21 with the plasmid pDI-CJ_{FA440} was designated as LSC-pDI-CJ_{FA440};
Specifically, the recombinant halophile obtained by transforming halophile Halomonas aydingkolgenesis M1 with the plasmid pDI-CJ_{FA440} was designated as M1-pDI-CJ_{FA440}.

### (3) Shake Flask Fermentation Study

The recombinant halophile strains were separately inoculated into 20 mL of LB60 culture medium (containing 25 μg/mL of chloramphenicol). After 10-12 h of incubation, the strains were transferred into a new LB60 culture medium (containing 25 μg/mL of chloramphenicol) of 20 mL at a volume proportion of 1%, and incubated for 8-12 h to give the shake flask fermentation seed solution.

2.5 mL of the fermentation seed solution was added into a 500-mL Erlenmeyer flask containing 47.5 mL of 60 LB fermentation medium for shake flask study. The chloramphenicol concentration was 25 μg/mL, the AHL concentration was 100×10⁻⁴ mM, the IPTG concentration was 200 mg/L, the concentration of sodium hexanoate or hexanoic acid was 5 g/L, the shaker temperature was 37° C., and the rotation speed was 200 rpm. After incubation for 48 hours, the cell dry weight and the PHBHHx content were determined. The groups were tested in triplicate, and the means were taken as the results, as shown in Table 2.

TABLE 2
PHBHHx production in recombinant halophiles containing
inducible phaC-phaJ expression plasmid module

| | | | Cell dry | PHA | 3HHx |
|---|---|---|---|---|---|
| Serial | | Carbon | weight | content | content |
| No. | Strain name | source | (g/L) | (wt %) | (mol %) |
| 1 | TD-pDI-CJ_{FA440} | Hexanoic | 2.78 ± 0.17 | 9.32 ± 1.01 | 4.38 ± 0.11 |
| | | acid | | | |
| 2 | | Sodium | 7.98 ± 0.21 | 30.11 ± 1.30 | 10.76 ± 0.61 |
| | | hexanoate | | | |
| 3 | TDC-pDI-CJ_{FA440} | Hexanoic | 2.54 ± 0.11 | 8.33 ± 1.14 | 6.58 ± 0.14 |
| | | acid | | | |
| 4 | | Sodium | 7.70 ± 0.30 | 28.30 ± 2.30 | 12.86 ± 0.80 |
| | | hexanoate | | | |
| 5 | LS-pDI-CJ_{FA440} | Hexanoic | 2.01 ± 0.25 | 8.43 ± 0.76 | 5.81 ± 0.24 |
| | | acid | | | |
| 6 | | Sodium | 7.85 ± 0.31 | 27.31 ± 1.01 | 11.22 ± 0.41 |
| | | hexanoate | | | |
| 7 | LSC-pDI-CJ_{FA440} | Hexanoic | 2.14 ± 0.11 | 7.33 ± 0.95 | 7.18 ± 0.14 |
| | | acid | | | |
| 8 | | Sodium | 8.10 ± 0.21 | 25.52 ± 1.31 | 13.67 ± 0.52 |
| | | hexanoate | | | |
| 9 | M1-pDI-CJ_{FA440} | Hexanoic | 2.94 ± 0.21 | 6.13 ± 0.65 | 8.17 ± 0.94 |
| | | acid | | | |
| 10 | | Sodium | 7.11 ± 0.81 | 20.52 ± 1.71 | 12.77 ± 1.53 |
| | | hexanoate | | | |
| 11 | TD-pDI-CJ_{4AK4} | Hexanoic | 2.52 ± 0.15 | 8.39 ± 1.21 | 3.94 ± 0.17 |
| | | acid | | | |
| 12 | | Sodium | 7.42 ± 0.18 | 27.11 ± 1.08 | 9.78 ± 0.51 |
| | | hexanoate | | | |
| 13 | TDC-pDI-CJ_{4AK4} | Hexanoic | 2.36 ± 0.19 | 8.63 ± 1.04 | 6.98 ± 0.24 |
| | | acid | | | |
| 14 | | Sodium | 7.19 ± 0.23 | 25.47 ± 1.30 | 11.96 ± 0.90 |
| | | hexanoate | | | |
| 15 | LS-pDI-CJ_{4AK4} | Hexanoic | 2.98 ± 0.27 | 9.43 ± 0.66 | 7.82 ± 0.34 |
| | | acid | | | |
| 16 | | Sodium | 7.95 ± 0.21 | 29.21 ± 1.21 | 10.12 ± 0.71 |
| | | hexanoate | | | |
| 17 | LSC-pDI-CJ_{4AK4} | Hexanoic | 2.09 ± 0.17 | 7.03 ± 0.75 | 7.88 ± 1.14 |
| | | acid | | | |
| 18 | | Sodium | 8.21 ± 0.71 | 23.42 ± 1.51 | 14.67 ± 0.92 |
| | | hexanoate | | | |
| 19 | M1-pDI-CJ_{4AK4} | Hexanoic | 2.62 ± 0.31 | 6.30 ± 0.76 | 8.07 ± 1.24 |
| | | acid | | | |
| 20 | | Sodium | 7.01 ± 1.21 | 19.27 ± 1.31 | 11.97 ± 1.37 |
| | | hexanoate | | | |

The results show that all 10 recombinant halophile strains can produce PHBHHx with hexanoic acid or sodium hexanoate as the related carbon source, and sodium hexanoate is a preferred carbon source compared with hexanoic acid, indicating that the effectiveness and the broad spectrum of PHBHHx production by introducing phaC-phaJ genes are confirmed sufficiently.

### Example 2: Construction of Recombinant Halophile Containing Constitutive phaC-phaJ Expression Plasmid Module for PHBHHx Production

In Example 1, the use of the inducible promoter required additional expensive inducers during the production of PHBHHx by fermentation, which increased the production cost. In order to avoid the use of inducers, a constitutive promoter was used to replace the inducible promoter so as to eliminate the need for expensive inducers. Meanwhile, in order to optimize and screen the constitutive promoter, the present invention further constructed a dual-inducible expression system. Furthermore, a rational relationship between the intensity of the inducible promoter and the intensity of the constitutive promoter was established through the dual-inducible expression system, and a mathematical basis was laid for optimizing and screening the constitutive promoter. The specific implementation procedures are as follows:

### (1) Construction of Dual-Inducible Characterization Plasmid

Based on the plasmid pDI-CJ_{FA440} in Example 1, a dual-inducible expression intensity characterization plasmid was constructed by replacing the PHA synthetase PhaC and the enoyl-coenzyme A hydratase PhaJ with green fluorescent protein (GFP, SEQ ID NO: 25) and red fluorescent protein (RFP, SEQ ID NO: 26), respectively, and the plasmid was designated as: pDI-dfp.

Specifically, the recombinant halophile obtained by transforming PHA synthetase-deficient halophile H. bluephagenesis TD01 with the plasmid pDI-dfp by E. coli S17-1 conjugation was designated as TDC-pDI-dfp.

The halophile TDC-pDI-dfp was cultured in a 2-mL deep-well plate system, and AHL and IPTG inducers at different concentrations were separately added. The expression level of the fluorescent proteins at corresponding concentrations was determined by flow cytometer.

Specifically, the following preferred AHL concentrations were selected: 0 mM, 0.1×10⁻⁴ mM, 0.5×10⁻⁴ mM, 1×10⁻⁴ mM, 5×10⁻⁴ mM, 10×10⁻⁴ mM, 50×10⁻⁴ mM, and 100×10⁻⁴ mM;
Specifically, the following preferred IPTG concentrations were selected: 0 mg/L, 0.5 mg/L, 1 mg/L, 5 mg/L, 10 mg/L, 20 mg/L, 100 mg/L, 200 mg/L, and 2000 mg/L;
The measurement results are shown in FIGS. 2-3.
   (2) Establishment of Inducible phaC and phaJ Expression Level Matrix and Shake Flask Fermentation Study:

AHL and IPTG inducer combinations at different concentrations were added to the shake flask fermentation process of the recombinant halophile strain TDC-pDI-CJ_{FA440} from Example 1. The influence of phaC and phaJ expression intensity on producing PHBHHx was determined by measuring the cell dry weight and PHBHHx content.

The recombinant halophile strain TDC-pDI-CJ_{FA440} was inoculated into 20 mL of LB60 culture medium (containing 25 μg/mL of chloramphenicol). After 10-12 h of incubation, the strain was transferred into a new LB60 culture medium (containing 25 μg/mL of chloramphenicol) of 20 mL at a volume proportion of 1%, and incubated for 8-12 h to give the shake flask fermentation seed solution.

2.5 mL of the fermentation seed solution was added into a 500-mL Erlenmeyer flask containing 47.5 mL of 60 LB fermentation medium for shake flask study. The chloramphenicol concentration was 25 μg/mL, AHL and IPTG were added at different concentrations, the shaker temperature was 37° C., and the rotation speed was 200 rpm. 20 g/L glucose and 5 g/L sodium hexanoate were used as the mixed carbon source for the fermentation.

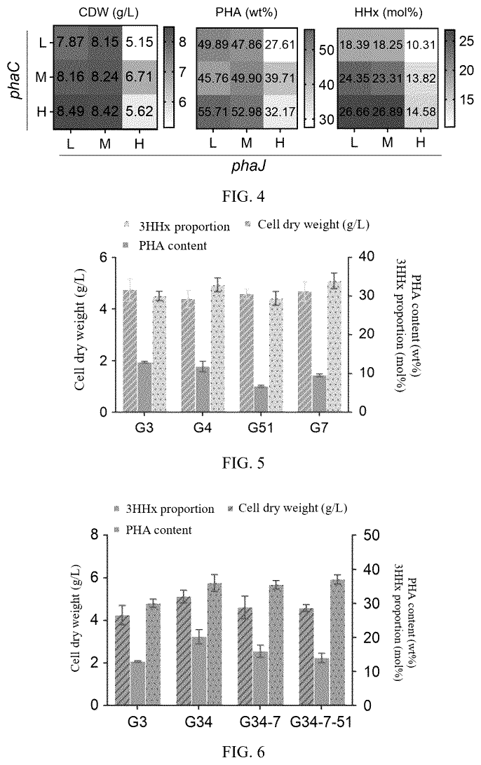

Specifically, three preferred AHL concentrations were selected: 1×10⁻⁴ mM (L), 10×10⁻⁴ mM (M), and 100×10⁻⁴ mM (H);
Specifically, three preferred IPTG concentrations were selected: 20 mg/L (L), 100 mg/L (M), and 200 mg/L (H);
Specifically, the number of the AHL and IPTG concentration combinations was: 3×3=9;
After incubation for 48 hours, the cell dry weight and the PHBHHx content were determined. The groups were tested in triplicate, and the means were taken as the results, as shown in FIG. 4. The results show that phaC and phaJ with different expression levels have a great influence on the PHBHHx producing by the recombinant strain. Among them, the combinations of the phaJ gene at low and medium expression levels and the phaC gene at low, medium and high expression levels demonstrated good results (cell dry weight, PHA content, and 3HHx proportion).
   (3) Construction of Constitutive phaC-phaJ Expression Plasmid Module

The expression level of the porin gene mutant promoter library is characterized in Stimulus response-based fine-tuning of polyhydroxyalkanoate pathway in Halomonas (Ye, et. al., Metabolic Engineering, 2020.). Based on the expression level of the preferred inducible promoters in this example, constitutive Pₚₒᵣᵢₙ promoters corresponding to the expression levels were selected.

Specifically, based on the pDI-CJ_{FA440} promoter constructed in Example 1, the AHL-inducible promoter and the IPTG-inducible promoter were replaced with Pₚₒᵣᵢₙ₅₈ and Pₚₒᵣᵢₙ₆₈ promoters, respectively, and the constructed constitutive phaC-phaJ expression plasmid was designated as: pDC-CJ_{FA440}.

### (4) Shake Flask Fermentation Study

By E. coli S17-1 binding assay, some halophiles were transformed with the plasmid pDC-CJ_{FA440}.

Specifically, the recombinant halophile obtained by transforming halophile H. bluephagenesis TD01 with the plasmid pDC-CJ_{FA440} was designated as TD-pDC-CJ_{FA440}.

Specifically, the recombinant halophile obtained by transforming PHA synthetase-deficient halophile H. bluephagenesis TD01 with the plasmid pDC-CJ_{FA440} was designated as TDC-pDC-CJ_{FA440}.

Specifically, the recombinant halophile obtained by transforming halophile H. campaniensis LS21 with the plasmid pDC-CJ_{FA440} was designated as LS-pDC-CJ_{FA440}. Specifically, the recombinant halophile obtained by transforming PHA synthetase-deficient halophile H. campaniensis LS21 with the plasmid pDC-CJ_{FA440} was designated as LSC-pDC-CJ_{FA440}.

The recombinant halophile strain was inoculated into 20 mL of LB60 culture medium (containing 25 μg/mL of chloramphenicol). After 10-12 h of incubation, the strain was transferred into a new LB60 culture medium (containing 25 μg/mL of chloramphenicol) of 20 mL at a volume proportion of 1%, and incubated for 8-12 h to give the shake flask fermentation seed solution.

2.5 mL of the fermentation seed solution was added into a 500-mL Erlenmeyer flask containing 47.5 mL of 60 LB fermentation medium for shake flask study. The chloramphenicol concentration was 25 μg/mL, the sodium hexanoate concentration was 7.5 g/L, the shaker temperature was 37° C., and the rotation speed was 200 rpm. The results are shown in Table 3.

TABLE 3
PHBHHx production in recombinant halophiles containing
constitutive phaC-phaJ expression plasmid module

| | | Cell dry | | |
|---|---|---|---|---|
| Serial | | weight | PHA content | 3HHx content |
| No. | Strain name | (g/L) | (wt %) | (mol %) |
| 1 | TD-pDC-CJ_{FA440} | 5.57 ± 0.22 | 13.57 ± 0.91 | 20.19 ± 2.11 |
| 2 | TDC-pDC-CJ_{FA440} | 5.36 ± 0.12 | 12.19 ± 0.81 | 29.89 ± 1.21 |
| 3 | LS-pDC-CJ_{FA440} | 5.67 ± 0.31 | 14.15 ± 0.98 | 20.11 ± 1.89 |
| 4 | LSC-pDC-CJ_{FA440} | 5.12 ± 0.22 | 13.11 ± 0.71 | 27.91 ± 2.01 |

The results show that the constitutive promoters can promote the expression of phaC and phaJ genes in different halophile strains.

Preferably, the construction of the constitutive phaC-phaJ expression plasmid module reduces the use of expensive inducers and optimizes the expression level of the genes.

### Example 3: Construction of Recombinant Halophile with Inactivated Key Genes in β-Oxidation Cycle Pathway for PHBHHx Production

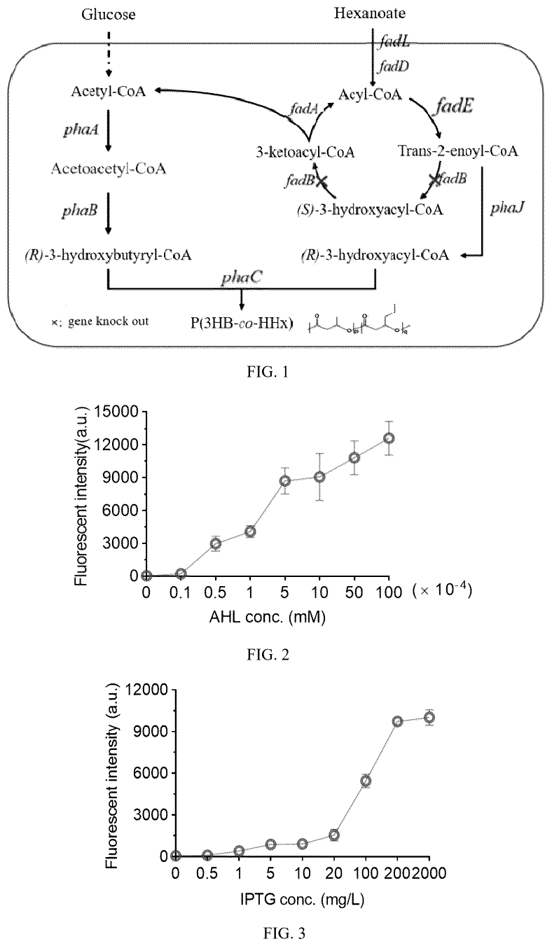

As can be seen from FIG. 1, the inactivation of key genes in the β-oxidation cycle pathway can further increase the efficiency of the conversion from substrate to 3HHx monomer, thereby increasing the molar proportion of 3HHx in PHBHHx. In this example, the molar proportion of 3HHx in PHBHHx was increased by knocking out the endogenous enoyl-coenzyme A hydratase fadB gene (SEQ ID NOs: 27 and 39-48) in the halophile β-oxidation cycle pathway.

The specific procedures are as follows:

(1) Knockout fadB Gene in Halophile

The genome annotation information of halophile H. bluephagenesis TD01 shows that the strain has 11 potential fadB genes. The target genes were knocked out by CRISPR/Cas9 genome editing method. Specifically, the construction methods of the plasmid containing the sgRNA and the recombinant template are as follows: The DNA fragments such as upstream and downstream 1000-bp homology arms, respectively, sgRNA expression modules and the like were inserted into the original expression plasmid pSEVA241 (containing kanamycin and spectinomycin resistance genes) by Gibson Assembly. The order of sequence arrangement in the plasmid is as follows: sgRNA expression module-upstream homology arm-downstream homology arm. Corresponding halophiles were transformed with the pSEVA241 plasmid for expressing the sgRNA and the recombinant template and the pQ08 plasmid for expressing Cas9 by E. coli S17-1 conjugation.

Mutants with fadB gene knockout were screened by primers designed by colony PCR, and were confirmed by gene sequencing. The colony PCR was conducted as per routines. Furthermore, strains with CRISPR/Cas9 plasmid loss were identified for the next round of genome editing by successive and multiple passages in liquid culture media of strains with successful genome editing and streaking culture on plates with spectinomycin resistance, chloramphenicol resistance, and no resistance.

Finally, colony PCR and gene sequencing confirmed that the fadB gene in the corresponding halophile genome had been knocked out.

Specifically, the recombinant halophile obtained by knocking out fadB genes in halophile H. bluephagenesis TD01 genome was designated as TDB.

Specifically, the recombinant halophile obtained by knocking out fadB genes in PHA synthetase-deficient halophile H. bluephagenesis TD01 genome was designated as TDCBn, wherein n denotes the fadB gene number.

(2) Construction of Recombinant Halophile with Heterologous phaC-phaJ Expression Plasmid Module

By E. coli S17-1 binding assay, a halophile was transformed with the plasmid pDC-CJ_{FA440} in Example 2.

Specifically, the recombinant halophile obtained by transforming halophile TDCB with the plasmid pDC-CJ_{FA440} was designated as TDCBn-pDC-CJ_{FA440}, wherein n denotes the fadB gene number.

### (3) Verification of PHBHHx Production Capability in Recombinant Halophiles by Shake Flask Study

The recombinant halophile strains were separately inoculated into 20 mL of LB60 culture medium (containing 25 μg/mL of chloramphenicol). After 10-12 h of incubation, the strains were transferred into a new LB60 culture medium (containing 25 μg/mL of chloramphenicol) of 20 mL at a volume proportion of 1%, and incubated for 8-12 h to give the fermentation seed solution.

2.5 mL of the fermentation seed solution was added into a 500-mL Erlenmeyer flask containing 47.5 mL of 60 LB fermentation medium for shake flask study. The chloramphenicol concentration was 25 μg/mL, the sodium hexanoate concentration was 7.5 g/L, the shaker temperature was 37° C., and the rotation speed was 200 rpm. After incubation for 48 hours, the cell dry weight and the PHBHHx content were determined. The groups were tested in triplicate, and the means were taken as the results, as shown in Table 4.

TABLE 4
PHBHHx production in recombinant halophiles
with inactivated fadB gene

| | | Cell dry | PHA | 3HHx |
|---|---|---|---|---|
| Serial | | weight | content | content |
| No. | Strain name | (g/L) | (wt %) | (mol %) |
| 1 | TDCB1-pDC-CJ_{FA440} | 5.13 ± 0.21 | 18.47 ± 0.29 | 49.05 ± 2.02 |
| 2 | TDCB2-pDC-CJ_{FA440} | 5.36 ± 0.12 | 11.19 ± 0.78 | 27.71 ± 1.11 |
| 3 | TDCB3-pDC-CJ_{FA440} | 5.19 ± 0.18 | 15.21 ± 0.18 | 26.05 ± 1.21 |
| 4 | TDCB4-pDC-CJ_{FA440} | 5.20 ± 0.11 | 14.77 ± 0.19 | 24.43 ± 1.53 |
| 5 | TDCB5-pDC-CJ_{FA440} | 5.25 ± 0.49 | 13.27 ± 0.21 | 25.08 ± 1.98 |
| 6 | TDCB6-pDC-CJ_{FA440} | 5.45 ± 0.21 | 14.62 ± 1.28 | 23.05 ± 1.92 |
| 7 | TDCB7-pDC-CJ_{FA440} | 5.10 ± 1.01 | 12.37 ± 1.31 | 28.15 ± 1.21 |
| 8 | TDCB8-pDC-CJ_{FA440} | 5.08 ± 0.11 | 13.26 ± 1.39 | 27.08 ± 1.92 |
| 9 | TDCB9-pDC-CJ_{FA440} | 5.09 ± 0.31 | 19.27 ± 0.32 | 22.09 ± 1.98 |
| 10 | TDCB10-pDC-CJ_{FA440} | 5.39 ± 1.18 | 12.27 ± 1.31 | 23.95 ± 1.02 |
| 11 | TDCB11-pDC-CJ_{FA440} | 5.21 ± 1.21 | 15.49 ± 1.09 | 24.05 ± 1.28 |

By comparison with the shake flask fermentation data of halophile TD-pDC-CJ_{FA440} in Example 2, it is found that fadB1 is a key gene in the β-oxidation cycle pathway in halophile H. bluephagenesis and derivatives thereof. The inactivation of fadB1 can improve the molar proportion of 3HHx monomer in PHBHHx.

### Example 4: PHBHHx Production in Recombinant Halophiles with Single-Copy phaC-phaJ Functional Module Expression at Different Sites in Genome

In the fermentation process, the use of plasmid expression functional module requires a large amount of antibiotics, thus increasing the production cost and the post-treatment difficulty of the fermentation liquor. In the shake flask fermentation studies in Example 1, Example 2, and Example 3 of the present invention, exogenous antibiotics were added. Therefore, by the expression sites in the halophile genome were screened, and the phaC-phaJ functional module was separately integrated into the designated sites in the genome by CRISPR/Cas9 gene editing, such that the recombinant halophile can stably produce PHBHHx without adding antibiotics.

The specific procedures are as follows:

### (1) Selection of Suitable Expression Sites in Halophile Genome

In this example, four functional module integration sites were preferred from the halophile genome for subsequent studies.

Specifically, the preferred halophile was the PHA synthetase-deficient halophile H. bluephagenesis in Example 1, designated as TDC.

Specifically, based on the previous transcriptome data of the strain, the four selected genomic integration sites were: G3 (see SEQ ID NO: 28 for the guide RNA sequence), G4 (see SEQ ID NO: 29 for the guide RNA sequence), G7 (see SEQ ID NO: 30 for the guide RNA sequence), and G51 (see SEQ ID NO: 31 for the guide RNA sequence). Specifically, the preferred phaC-phaJ functional module was: “Pₚₒᵣᵢₙ₅₈-RBS1-phaC_{FA440}-T1-Pₚₒᵣᵢₙ₆₈-RBS2-phaJ_{FA440}-T2” sequence, as set forth in SEQ ID NO: 32.

(2) Construction of phaC-phaJ Functional Module Integration Plasmid

The target DNA sequence was integrated by CRISPR/Cas9 genome editing. Specifically, the construction methods of the plasmid containing the sgRNA and the recombinant template are as follows: The DNA fragments such as upstream and downstream 1000-bp homology arms, respectively, sgRNA expression modules, phaC-phaJ functional modules and the like were inserted into the original expression plasmid pSEVA241 (containing kanamycin and spectinomycin resistance genes) by Gibson Assembly. The order of sequence arrangement in the plasmid is as follows: sgRNA expression module-upstream homology arm-“phaC-phaJ functional module”-downstream homology arm.

Specifically, the sequence of the plasmid integrated at G3 site is set forth in SEQ ID NO: 33;
Specifically, the sequence of the plasmid integrated at G4 site is set forth in SEQ ID NO: 34;
Specifically, the sequence of the plasmid integrated at G7 site is set forth in SEQ ID NO: 35;
Specifically, the sequence of the plasmid integrated at G51 site is set forth in SEQ ID NO: 36;
Halophile strain TDC was transformed with the pSEVA241 plasmid for expressing the sgRNA and the recombinant template and the pQ08 plasmid for expressing Cas9 by E. coli S17-1 conjugation.

Mutants with phaC-phaJ functional module knockin were screened by primers designed by colony PCR, and were confirmed by gene sequencing. The colony PCR was conducted as per routines. Furthermore, strains with CRISPR/Cas9 plasmid loss were identified for the next round of genome editing by successive and multiple passages in liquid culture media of strains with successful genome editing and streaking culture on plates with spectinomycin resistance, chloramphenicol resistance, and no resistance.

Finally, colony PCR and DNA sequencing confirmed that the phaC-phaJ functional module was knocked into the different sites of halophile TDC genome.

Specifically, the recombinant halophile obtained by phaC-phaJ functional module knockin at G3 site in halophile TDC genome was designated as TDC-G3.

Specifically, the recombinant halophile obtained by phaC-phaJ functional module knockin at G4 site in halophile TDC genome was designated as TDC-G4.

Specifically, the recombinant halophile obtained by phaC-phaJ functional module knockin at G7 site in halophile TDC genome was designated as TDC-G7.

Specifically, the recombinant halophile obtained by phaC-phaJ functional module knockin at G51 site in halophile TDC genome was designated as TDC-G51.

### (3) Verification of PHBHHx Production Capability in Recombinant Halophiles by Shake Flask Study

The recombinant halophile strains in this example were separately inoculated into 20 mL of LB60 culture medium. After 10-12 h of incubation, the strains were transferred into a new LB60 culture medium of 20 mL at a volume proportion of 1%, and incubated for 8-12 h to give the fermentation seed solution. 2.5 mL of the fermentation seed solution was added into a 500-mL Erlenmeyer flask containing 47.5 mL of 60 LB fermentation medium for shake flask study. The sodium hexanoate concentration was 7.5 g/L, the shaker temperature was 37° C., and the rotation speed was 200 rpm. After incubation for 48 hours, the cell dry weight and the PHBHHx content were determined. The groups were tested in triplicate, and the means were taken as the results, as shown in FIG. 5. The results show that the integration of the phaC-phaJ functional module into the preferred genomic site in the halophile can achieve stable PHBHHx production independent of antibiotics.

Preferably, the expression efficiency order of the integration sites is: G3>G4>G7>G51.

### Example 5: PHBHHx Production in Recombinant Halophiles with Multi-Copy phaC-phaJ Functional Module Expression at Different Sites in Genome

In Example 4, the integration of the phaC-phaJ functional module into the preferred genomic site in the halophile achieved stable PHBHHx production independent of antibiotics. Furthermore, in this example, by sequentially integrating the phaC-phaJ functional module into designated sites in the genome to increase the copy number of the phaC-phaJ functional module in the genome, the ability of the recombinant halophile to produce PHBHHx can be further improved.

The specific procedures are as follows:

(1) Construction of Recombinant Halophiles with Multi-Copy phaC-phaJ Functional Module

The phaC-phaJ functional module constructed in Example 4 was integrated at different sites in the halophile to give recombinant halophiles with different phaC-phaJ functional module copy numbers in the genome.

The target DNA sequence was integrated by CRISPR/Cas9 genome editing. Specifically, the construction methods of the plasmid containing the sgRNA and the recombinant template are as follows: The DNA fragments such as upstream and downstream 1000-bp homology arms, respectively, sgRNA expression modules, phaC-phaJ functional modules and the like were inserted into the original expression plasmid pSEVA241 (containing kanamycin and spectinomycin resistance genes) by Gibson Assembly. The order of sequence arrangement in the plasmid is as follows: sgRNA expression module-upstream homology arm-“phaC-phaJ functional module”-downstream homology arm.

Halophile strain TDC was transformed with the pSEVA241 plasmid for expressing the sgRNA and the recombinant template and the pQ08 plasmid for expressing Cas9 by E. coli S17-1 conjugation.

Mutants with phaC-phaJ functional module knockin were screened by primers designed by colony PCR, and were confirmed by gene sequencing. The colony PCR was conducted as per routines. Furthermore, strains with CRISPR/Cas9 plasmid loss were identified for the next round of genome editing by successive and multiple passages in liquid culture media of strains with successful genome editing and streaking culture on plates with spectinomycin resistance, chloramphenicol resistance, and no resistance.

Finally, colony PCR and DNA sequencing confirmed that the phaC-phaJ functional module was knocked into the recombinant halophile genome at a specific site. Furthermore, the copy number of the phaC-phaJ functional module in the genome of the recombinant halophile was sequentially increased by repeated CRISPR/Cas9 genome editing.

Specifically, the recombinant halophile obtained by phaC-phaJ functional module knockin at G3 site in halophile TDC genome was designated as TDC-G3. The recombinant halophile genome contained 1 copy of the phaC-phaJ functional module. Specifically, the recombinant halophile obtained by phaC-phaJ functional module knockin at G3 and G4 sites in halophile TDC genome was designated as TDC-G34. The recombinant halophile genome contained 2 copies of the phaC-phaJ functional module.

Specifically, the recombinant halophile obtained by phaC-phaJ functional module knockin at G3, G4, and G7 sites in halophile TDC genome was designated as TDC-G34-7. The recombinant halophile genome contained 3 copies of the phaC-phaJ functional module.

Specifically, the recombinant halophile obtained by phaC-phaJ functional module knockin at G3, G4, G7, and G51 sites in halophile TDC genome was designated as TDC-G34-7-51. The recombinant halophile genome contained 4 copies of the phaC-phaJ functional module.

### (2) Verification of PHBHHx Production Capability in Recombinant Halophiles by Shake Flask Study

The recombinant halophile strains in this example were separately inoculated into 20 mL of LB60 culture medium. After 10-12 h of incubation, the strains were transferred into a new LB60 culture medium of 20 mL at a volume proportion of 1%, and incubated for 8-12 h to give the fermentation seed solution.

2.5 mL of the fermentation seed solution was added into a 500-mL Erlenmeyer flask containing 47.5 mL of 60 LB fermentation medium for shake flask study. The sodium hexanoate concentration was 7.5 g/L, the shaker temperature was 37° C., and the rotation speed was 200 rpm. After incubation for 48 hours, the cell dry weight and the PHBHHx content were determined. The groups were tested in triplicate, and the means were taken as the results, as shown in FIG. 6. The results show that increasing the copy number of the phaC-phaJ functional module which integrated into the halophile genome can increase the ability of the strain to produce PHBHHx. The 2-copy group exhibited a higher cell dry weight and a higher PHA content than the other groups, while the 4-copy group had a higher 3HHx proportion than the others.

### Example 6: PHBHHx Production in Recombinant Halophiles at Low Salinity

The halophile requires a high concentration of sodium chloride (60 g/L NaCl) to maintain the osmotic pressure during the fermentative production process, which, however, also causes a very complicated treatment process for high-salinity wastewater in the fermentation post-treatment, and directly increases the overall production cost of the product. In the present invention, the production cost can be directly reduced by using halophile tolerant to low salinity as a chassis strain to produce PHBHHx.

The specific procedures are as follows:

### (1) Construction of Recombinant Low-Salinity-Resistant Halophile

A low-salinity-resistant halophile was transformed with the constitutive phaC-phaJ expression plasmid (pDC-CJ_{FA440}) in Example 2 by E. coli S17-1 conjugation to give a recombinant low-salinity-resistant halophile.

Specifically, the preferred low-salinity-resistant halophile is Halomonas bluephagenesis TDH4AB.

Specifically, the recombinant halophile obtained by transforming halophile Halomonas bluephagenesis TDH4AB with the plasmid pDC-CJ_{FA440} was designated as TDH4AB-pDC-CJ_{FA440}.

### (2) Verification of PHBHHx Production Capability in Recombinant Low-Salinity-Resistant Halophile by Shake Flask Study

The recombinant halophile strain TDH4AB-pDC-CJ_{FA440} in this example was inoculated into 20 mL of LB culture medium. After 10-12 h of incubation, the strain was transferred into a new LB culture medium of 20 mL at a volume proportion of 1%, and incubated for 8-12 h to give the fermentation seed solution.

2.5 mL of the fermentation seed solution was added into a 500-mL Erlenmeyer flask containing 47.5 mL of LB fermentation medium (NaCl concentration: 10 g/L) for shake flask study. The chloramphenicol concentration was 25 μg/mL, the sodium hexanoate concentration was 7.5 g/L, the shaker temperature was 37° C., and the rotation speed was 200 rpm. After incubation for 48 hours, the cell dry weight and the PHBHHx content were determined. The groups were tested in triplicate, and the means were taken as the results, as shown in Table 5.

TABLE 5
PHBHHx production in recombinant
low-salinity-resistant halophile

| | | Cell dry | PHA | 3HHx |
|---|---|---|---|---|
| Serial | | weight | content | content |
| No. | Strain name | (g/L) | (wt %) | (mol %) |
| 1 | TDH4AB-pDC- | 5.36 ± 1.09 | 11.19 ± 1.21 | 18.27 ± 1.34 |
| | CJ_{FA440} | | | |

The result shows that the PHBHHx can be produced by using related carbon sources in the recombinant low-salinity-resistant halophile.

Preferably, the use of the low-salinity-resistant halophile Halomonas bluephagenesis TDH4AB as the chassis strain reduces the amount of NaCl and thus simplifies the fermentation wastewater treatment process.

### Example 7: PHBHHx Production in Recombinant Halophile in Fermenter Using Sodium Hexanoate as Sole Carbon Source

The laboratory fermenter study is a validation at the shake flask level and a basis for large-scale fermentative production. The recombinant halophile TDC-G34 constructed in Example 5 was used as the fermentation strain to produce PHBHHx in a 7-L fermenter using sodium hexanoate as a sole carbon source.

The specific procedures are as follows:

The recombinant halophile TDC-G34 was inoculated into 20 mL of LB60 culture medium. After 12-16 h of incubation, the strains were transferred into a new LB60 culture medium at a volume proportion of 1%, and incubated for 8-12 h. 300 mL of seed solution was prepared as the seeding solution for the 7-L bioreactor (NBS Bioflo3000).

2.7 L of basis medium was prepared. The composition of the basis material was: sodium chloride (135 g), potassium chloride (15 g), yeast extract (30 g), urea (9 g), disodium citrate (7.8 g), anhydrous magnesium sulfate (0.6 g), potassium dihydrogen phosphate (15.6 g), component III (30 mL), and component IV (3 mL).

The amount of dissolved oxygen was adjusted by stirring and aeration during the fermentation process; the pH value of the culture medium was set to 8.5 and automatically adjusted by NaOH; the temperature was set at 37° C. and was automatically controlled by the reactor.

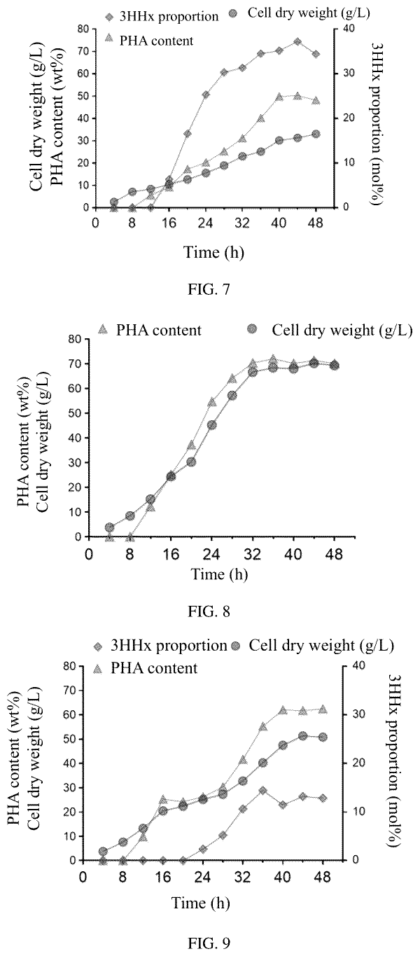

After 8 h of fermentation, sodium hexanoate was fed batch, and the sodium hexanoate concentration was determined on line by HPLC and controlled at not more than 7.5 g/L. The results are shown in FIG. 7.

The results show that the molar proportion of 3HHx monomer in PHBHHx produced by the recombinant halophile was about 40 mol % after 48 h of fermentation.

Preferably, the recombinant halophile can produce PHBHHx containing a high molar proportion of 3HHx using sodium hexanoate as a sole carbon source.

### Example 8: PHA Production in Recombinant Halophile in Fermenter Using Glucose as Sole Carbon Source

The recombinant halophile TDC-G34 constructed in Example 5 was used as the fermentation strain to produce PHA in a 7-L fermenter using glucose as the sole carbon source.

The specific procedures are as follows:

The recombinant halophile TDC-G34 was inoculated into 20 mL of LB60 culture medium. After 12-16 h of incubation, the strains were transferred into a new LB60 culture medium at a volume proportion of 1%, and incubated for 8-12 h. 300 mL of seed solution was prepared as the seeding solution for the 7-L bioreactor (NBS Bioflo3000).

2.7 L of basis medium was prepared. The composition of the basis material was: glucose (60 g), sodium chloride (135 g), potassium chloride (15 g), yeast extract (30 g), urea (9 g), disodium citrate (7.8 g), anhydrous magnesium sulfate (0.6 g), potassium dihydrogen phosphate (15.6 g), component III (30 mL), and component IV (3 mL).

A feed I medium containing glucose (200 g), yeast extract (8 g), and urea (32 g) was prepared.

A feed II medium containing glucose (200 g), yeast extract (4 g), and urea (28 g) was prepared.

The amount of dissolved oxygen was adjusted by stirring and aeration during the fermentation process; the pH value of the culture medium was set to 8.5 and automatically adjusted by NaOH; the temperature was set at 37° C. and was automatically controlled by the reactor.

After 8 h of fermentation, the feed I and feed II media were fed batch in sequence, and the content of the residual glucose in the fermenter was immediately determined by a glucometer so as to control the concentration of the residual glucose at 5-10 g/L. The results are shown in FIG. 8.

The results show that the PHA produced by the recombinant halophile TDC-G34 using glucose as a sole carbon source after 48 h of fermentation was P3HB, and no PHBHHx was produced.

### Example 9: PHBHHx Production in Recombinant Halophile in Fermenter Using Mixed Carbon Source

The recombinant halophile TDC-G34 constructed in Example 5 was used as the fermentation strain to produce PHBHHx in a 7-L fermenter using a mixed carbon source containing glucose and sodium hexanoate.

The specific procedures are as follows:

The recombinant halophile TDC-G34 was inoculated into 20 mL of LB60 culture medium. After 12-16 h of incubation, the strains were transferred into a new LB60 culture medium at a volume proportion of 1%, and incubated for 8-12 h. 300 mL of seed solution was prepared as the seeding solution for the 7-L bioreactor (NBS Bioflo3000).

2.7 L of basis medium was prepared. The composition of the basis material was: glucose (60 g), sodium chloride (135 g), potassium chloride (15 g), yeast extract (30 g), urea (9 g), disodium citrate (7.8 g), anhydrous magnesium sulfate (0.6 g), potassium dihydrogen phosphate (15.6 g), component III (30 mL), and component IV (3 mL).

A feed I medium containing glucose (200 g), yeast extract (8 g), and urea (32 g) was prepared.

A feed II medium containing glucose (200 g), yeast extract (4 g), and urea (28 g) was prepared.

The amount of dissolved oxygen was adjusted by stirring and aeration during the fermentation process; the pH value of the culture medium was set to 8.5 and automatically adjusted by NaOH; the temperature was set at 37° C. and was automatically controlled by the reactor.

After 8 h of fermentation, the feed I and feed II media were fed batch in sequence, and the content of the residual glucose in the fermenter was immediately determined by a glucometer so as to control the concentration of the residual glucose at 5-10 g/L.

At the same time, after 8 h of fermentation, sodium hexanoate was fed batch, and the sodium hexanoate concentration was determined on line by HPLC and controlled at not more than 7.5 g/L. The results are shown in FIG. 9.

The results show that the molar proportion of 3HHx in PHBHHx was about 12 mol % after 48 h of fermentation.

Preferably, in combination with Examples 7 and 8, the molar proportion of 3HHx in PHBHHx can be controlled by adjusting the ratio of sodium hexanoate to glucose.

### Example 10: PHBHHx Production in Recombinant Halophile in Shake Flask Using Mixed Carbon Source

The recombinant halophile TDC-G34 constructed in Example 5 was used as the fermentation strain to produce PHBHHx in shake flasks using a mixed carbon source containing glucose and sodium hexanoate.

The specific procedures are as follows:

The recombinant halophile TDC-G34 was inoculated into 20 mL of LB60 culture medium. After 10-12 h of incubation, the strain was transferred into a new LB60 culture medium of 20 mL at a volume proportion of 1%, and incubated for 8-12 h to give the shake flask fermentation seed solution.

2.5 mL of the shake flask fermentation seed solution was added into a 500-mL Erlenmeyer flask containing 47.5 mL of 60 LB fermentation medium for culture. The final concentrations of sodium hexanoate and glucose are shown in Table 6. The shaker temperature was 37° C. and the rotation speed was 200 rpm. After incubation for 48 hours, the cell dry weight and the PHBHHx content were determined. The groups were tested in triplicate, and the means were taken as the results. The results of the PHBHHx production in strain TDC-G34 using mixed carbon sources are shown in Table 6, and it was further confirmed that the molar proportion of 3HHx in PHBHHx can be controlled by adjusting the ratio of sodium hexanoate to glucose, and the adjustable range of the molar proportion of 3HHx in PHBHHx is: 0-38.17 mol %.

TABLE 6
PHBHHx production in strain TDC-
G34 using mixed carbon sources

| | Mixed carbon source | | | | |
|---|---|---|---|---|---|
| | (g/L) | | Cell dry | PHA | 3HHx |
| Serial | Sodium | | weight | content | content |
| No. | hexanoate | Glucose | (g/L) | (wt %) | (mol %) |
| 1 | 7.5 | 0 | 5.12 ± 0.37 | 17.80 ± 1.00 | 38.17 ± 2.30 |
| 2 | 7.5 | 10 | 6.60 ± 0.21 | 35.20 ± 2.10 | 13.94 ± 2.10 |
| 3 | 7.5 | 20 | 7.80 ± 0.30 | 50.20 ± 1.80 | 10.01 ± 1.10 |
| 4 | 7.5 | 30 | 7.50 ± 0.15 | 48.20 ± 1.20 | 8.21 ± 1.30 |
| 5 | 0 | 30 | 12.5 ± 0.21 | 50.13 ± 0.82 | 0 |

The preferred embodiments of the present invention are described in detail above, however, the present invention is not limited to specific details in the embodiments described in detail above. Within the scope of the technical conception of the present invention, various simple modifications can be made to the embodiments of the present invention, all of which will fall within the protection scope of the present invention.

1. A recombinant halophile, wherein the recombinant halophile expresses an exogenous phaC gene and/or phaJ gene, and/or, the recombinant halophile does not express a key protein of the β-oxidation cycle pathway or expresses proteins with no functions or reduced functions.

2. The recombinant halophile according to claim 1, wherein the recombinant halophile is capable of expressing an additional polymerase capable of polymerizing 3-hydroxybutyric acid and 3-hydroxyhexanoic acid.

3. The recombinant halophile according to claim 1, wherein the phaC gene and/or phaJ gene are regulated by an inducible promoter and/or a constitutive promoter.

4. The recombinant halophile according to claim 3, wherein the constitutive promoter includes a wild-type Pₚₒᵣᵢₙ or a mutant thereof;
the Pₚₒᵣᵢₙ mutant comprises one or two or more of mutant Pₚₒᵣᵢₙ₅₈, mutant Pₚₒᵣᵢₙ₄₂, mutant Pₚₒᵣᵢₙ₆₈, mutant Pₚₒᵣᵢₙ₂₇₈, mutant Pₚₒᵣᵢₙ₁₉₄, mutant Pₚₒᵣᵢₙ₂₂₁, or mutant Pₚₒᵣᵢₙ₂₀₃.

5. The recombinant halophile according to claim 3, wherein the inducible promoter includes a Pₗᵤₓ promoter and/or a P_{lac} promoter.

6. The recombinant halophile according to claim 1, wherein the phaC gene and/or phaJ gene is expressed on a plasmid, and/or, expressed by integration into the genome.

7. The recombinant halophile according to claim 6, wherein the recombinant halophile comprises a single copy or two or more copies of the phaC gene and/or phaJ gene.

8. The recombinant halophile according to claim 1, wherein the key protein of the β-oxidation cycle pathway includes enoyl-coenzyme A hydratase.

9. The recombinant halophile according to claim 1, wherein the recombinant halophile does not express a PHA synthetase or expresses a protein with no functions or reduced functions.

10. The recombinant halophile according to claim 1, wherein the recombinant halophile includes Halomonas bluephagenesis or a derivative thereof, Halomonas campaniensis or a derivative thereof, or Halomonas aydingkolgenesis or a derivative thereof.

11. The recombinant halophile according to claim 1, wherein the recombinant halophile produces PHBHHx.

12. A method for producing the recombinant halophile according to claim 1, comprising introducing into a halophile one or two of the following:
1) a phaC gene and/or phaJ gene;
2) an sgRNA and/or a gene encoding Cas9 protein, the sgRNA targeting an fadB gene.

13. The method according to claim 12, wherein the target site sequence of the sgRNA targeting the fadB gene comprises SEQ ID NO: 37.

14. A vector, comprising:
1) a phaC gene and/or phaJ gene; and/or
2) an sgRNA and/or a gene encoding Cas9 protein, the sgRNA targeting an fadB gene.

15. The vector according to claim 14, wherein the target site sequence of the sgRNA targeting the fadB gene comprises SEQ ID NO: 37.

16. A method for producing PHBHHx, comprising: fermentatively culturing the recombinant halophile according to claim 1.

17. The method according to claim 16, wherein the carbon source in the fermentation process includes one or two or more of hexanoic acid, a hexanoate, or glucose.

18. The method according to claim 16, wherein the method does not require sterilization.

19. A method for increasing the molar proportion of 3HHx monomer in PHBHHx produced by a halophile, comprising: fermentatively culturing the recombinant halophile according to claim 1, and/or adjusting the carbon source during the fermentation process.

20. The method according to claim 19, wherein the carbon source comprises one or two or more of hexanoic acid, a hexanoate, or glucose.
